(19) United States
(12) Patent Application Publication
(10) Pub. No.: US 2007/0207504 A1
(43) Pub. Date: Sep. 6, 2007

(54) Proteomic methods for the identification of differentiated adipose cells and adipose derived adult stem cells

(75) Inventors: Jeffrey M. Gimble, Baton Rouge, LA (US); Michael Lefevre, Baton Rouge, LA (US); James P. DeLany, Sewickley, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: The Board of Supervisors of Louisiana State University and Agricultural and Mechanical College; as represented by Pennington Biomedical Research Center, a component of the Louisiana State; University System duly organized and existing under the laws of the State of Louisiana.

(21) Appl. No.: 11/369,116

(22) Filed: Mar. 6, 2006

(51) Int. Cl. G01N 33/567; C12N 5/08

(52) U.S. Cl. 435/7.2; 435/366

(57) ABSTRACT

The present invention includes method for identifying, differentiating and distinguishing undifferentiated adipose-derived adult stem cells and differentiated adipose-derived adult stem cells using the proteomic profile of an adipose cell.

### BACKGROUND OF THE INVENTION

Obesity is a health problem of epidemic proportions. It is estimated that in 2000 over 60% of adults are overweight (BMI>25) and that 30% are obese (BMI>30); this compares to levels of 46% and 14%, respectively, in 1980. Obesity and increased adiposity are associated clinically with the onset of insulin resistance, dysfunctional glucose sensing and utilization, hypertension, and hypertriglyceridemia, all contributing to the pathologic sequelae of type 2 diabetes. Paradoxically, type 2-diabetes also occurs in patients with inherited or acquired forms of lipodystrophy or loss of adipose tissue depots (Garg, 2000, Am. J. Med., 108: 143-152; (Gougeon, et al., 2004, Antivir. Ther., 9: 161-177).

Lipodystrophy occurs through defects in genes associated with triglyceride metabolism or as a consequence of anti-retroviral therapy in HIV positive patients (Garg, 2000, Am. J. Med., 108: 143-152; Gougeon, et al., 2004, Antivir. Ther., 9: 161-177). Animal models confirm these clinical observations; multiple strains of transgenic mice with a lipodystrophic phenotype exhibit type 2 diabetes (Gavrilova, et al., 2000, J. Clin. Invest., 105:271-278; Shimomura, et al., 1998, Genes Dev., 12: 3182-3194; Shimomura, et al., 1999, Nature 401:73-76). Diabetes in these animals responds, not to insulin therapy, but to transplantation of subcutaneous adipose tissue or to leptin treatment (Gavrilova, et al., 2000, J. Clin. Invest., 105:271-278; Shimomura, et al., 1998, Genes Dev., 12: 3182-3194; Shimomura, et al., 1999, Nature 401:73-76). These clinical and experimental observations have led to the hypothesis that a failure in adipocyte differentiation is a critical etiologic factor leading to type 2 diabetes (Danforth, et al., 2000, Nat. Genet. 26: 13; Weber, et al., 2000, Am. J. Physiol. Regul. Integr. Comp. Physiol., 279: R936-43; Cederberg, et al., 2003, Curr. Mol. Med. 3: 107-125). Danforth and others postulate that in obese individuals, adipose tissue depots have already committed all of their stem cell reserves to the adipocyte lineage and have lost their capacity to create new adipocytic cells (Danforth, et al., 2000, Nat. Genet. 26: 13; Weber, et al., 2000, Am. J. Physiol. Regul. Integr. Comp. Physiol., 279: R936-43; Cederberg, et al., 2003, Curr. Mol. Med. 3: 107-125). In the face of excess energy balance, both obese and lipodystrophic individuals deposit triglycerides in ectopic sites, such as muscle and liver, thereby contributing to the metabolic dysfunction associated with type 2-diabetes (increased hepatic gluconeogenesis, skeletal muscle insulin resistance, abnormal pancreatic insulin secretion) (Danforth, et al., 2000, Nat. Genet. 26: 13). As a result of these findings, there has been a renewed interest in adipocyte progenitor cells as therapeutic targets and experimental models for studies of obesity and type 2-diabetes.

Murine cell models, most notably, the 3T3-L1 cell line, have been the basis for the majority of studies of adipogenesis at the transcriptional and protein levels. However, there is a growing concern that adipogenesis may differ between human and murine systems. For example, the resistin gene and its secreted protein product were first identified in the 3T3-L1 cells (Steppan, et al., 2001, Nature, 409: 307-312). Subsequent in vivo analysis in mice demonstrated an association between resistin levels, obesity, and type 2-diabetes (Steppan, et al., 2001, Nature, 409: 307-312). In contrast, clinical studies do not demonstrate a comparable association between serum resistin levels, obesity, and insulin resistance in non-obese and obese human subjects (Heilbronn, et al., 2004, J. Clin. Endocrinol. Metab., 89: 1844-1848). Likewise, the regulation of the agouti gene in adipose tissue differs between man and mouse (Smith, et al., 2003, Diabetes 52: 2914-2922). These discrepancies argue for the increased use of human pre-adipocyte cell models in exploratory research relating to obesity and type 2 diabetes.

Proteomic analyses of total cell lysates from murine 3T3-L1 adipocytes have identified between 8 and 100 protein features by one and two-dimensional gel electrophoresis/mass spectroscopy (Welsh, et al., 2004, Proteomics, 4: 1042-1051; Wilson-Fritch, et al., 2003, Mol. Cell. Biol., 23: 1085-1094; Brasaemle, et al., 2004 J. Biol. Chem., 279: 46835-46842; Choi, et al., 2004, Proteomics 4: 1840-8). However, previously detected proteins, especially those in murine systems differ significantly in expression patterns when compared to those of primary human adipocytes. In addition, previous studies have focused on the secreted proteins (Kratchmarova, et al., 2002, Mol. Cell. Proteomics. 1: 213-222) or lipid droplet associated proteins (Brasaemle, et al., 2004, J. Biol. Chem. 279: 46835-46842) in 3T3-L1 adipocytes.

The proteome of the caveolae and mitochondrial/nuclear fractions from human adipocytes has been reported (Aboulaich, et al., 2004, Biochem. J., 383(Pt 2): 237-248). β-actin, a number of annexins, G protein subunits, F1 ATPase subunits, and heat shock proteins, were identified.

Human adipose-derived adult stem cells offer an alternative in vitro model (Gimble, 2003, Expert Opinion in Biological Therapy 3: 705-713; Gimble and Guilak, 2003, Current Topics in Developmental Biology, 58: 137-160). These cells can be reproducibly isolated from liposuction aspirates through a procedure involving collagenase digestion, differential centrifugation, and expansion in culture. A single milliliter of tissue yields over 400,000 cells (Aust, et al., 2004, Cytotherapy 6: 1-8). The undifferentiated human adipocyte cells express a distinct immunophenotype based on flow cytometric analyses and, following induction, produce additional adipocyte specific proteins (Aust, et al., 2004, Cytotherapy 6: 1-8; 2001, J. Cell Physiol., 189: 54-63; Halvorsen, et al., 2001, Metabolism 50: 407-413; Sen, 2001, J. Cell. Biochem. 81: 312-319; Zuk, et al., 2002, Mol. Biol. Cell. 13: 4279-4295). The human adipose-derived adult stem cells display multipotentiality, with the capability of differentiating along the adipocyte, chondrocyte, myogenic, neuronal, and osteoblast lineages Aust, et al., 2004, Cytotherapy 6: 1-8; 2001, J. Cell Physiol., 189: 54-63; Halvorsen, et al., 2001, Metabolism 50: 407-413; Sen, 2001, J. Cell. Biochem. 81: 312-319; Zuk, et al., 2002, Mol. Biol. Cell. 13: 4279-4295; Ashjian, et al., 2003, Plast. Reconstr. Surg., 111: 1922-19231; Awad, et al., 2003, Tissue Engineering, 9: 1301-1312; Awad, et al., 2004, Biomaterials 25: 3211-3222; Halvorsen, et al., 2001, Tissue Eng., 7: 729-741; Hicok, et al., 2004, Tissue Engineering 10: 371-380; Mizuno, et al., 2002, Plast. Reconstr. Surg. 109: 199-209; Safford, et al., 2002, Biochem. Biophys. Res. Commun., 294: 371-379; Safford, et al., 2004, Experimental Neurology, 187: 319-328; Wickham, et al., 2003, Clin. Orthop., 412: 196-212; Winter, et al., 2003, Arthritis Rheum., 48: 418-429; Zuk, et al., 2001, Tissue Eng. 7: 211-28). In the presence of dexamethasone, insulin, isobutylmethylxanthine and a thiazolidinedione, the undifferentiated human adipocyte cells undergo adipogenesis; between 30% to 80% of the cells, based on flow cytometric methods, accumulate lipid vacuoles, which can be stained for neutral lipid with Oil Red O dye (Halvorsen, et al., 2001, Metabolism 50: 407-413; Sen, et al., 2001, J. Cell. Biochem., 81: 312-319).

The ability to identify proteins expressed on differentiated versus undifferentiated adipocytes, especially in therapeutically and biologically relevant primary human cells, is an important step in elucidating the mechanisms of several diseases associated with adipose tissue, including obesity and type 2 diabetes. The ability to identify proteins in differentiated versus undifferentiated adipocytes is also useful for identifying populations of multipotent adipose-derived adult stem cells. The present invention provides the means for identifying these proteins and cells.

### BRIEF SUMMARY OF THE INVENTION

The present invention comprises a method of identifying a differentiated adipose-derived adult stem cell, the method comprising comparing a proteomic profile of a first adipose-derived adult stem cell to a proteomic profile of a second adipose-derived adult stem cell, wherein the proteomic profile of the first adipose-derived adult stem cell comprises a protein that is specific for the first adipose-derived adult stem cell and is not upregulated in the proteomic profile of the second adipose-derived adult stem cell, thereby identifying a differentiated adipose-derived adult stem cell.

In one aspect of the present invention, the adipose-derived adult stem cell is a human adipose-derived adult stem cell.

In another aspect of the present invention, the protein is selected from the group consisting of a metabolism-related protein, a heat shock protein, a redox protein, a cytoskeletal protein, a serine protease inhibitor protein, and a protein degradation-related protein.

In yet another aspect of the present invention, the proteomic profile comprises at least two proteins specific for said differentiated adipose-derived adult stem cell.

In still another aspect of the present invention, the protein specific for said differentiated adipose-derived adult stem cell is upregulated about 2-fold compared to said second adipose-derived adult stem cell.

In another aspect of the present invention, the protein is selected from the group consisting of fatty acid binding protein-adipocyte, heat shock protein 20-like protein, heat shock protein β, heat shock protein 20, heat shock protein 27, heat shock protein 60, plasminogen activator inhibitor-1, pigmented epidermal derived factor, placental thrombin inhibitor, pregnancy zone protein, and protease C1 inhibitor.

The present invention comprises a method of identifying a differentiated adipose-derived adult stem cell, the method comprising comparing a proteomic profile of a first adipose-derived adult stem cell to a proteomic profile of a second adipose-derived adult stem cell, wherein the proteomic profile of the first adipose-derived adult stem cell comprises a protein that is specific for the first adipose-derived adult stem cell and is not down-regulated in the proteomic profile of the second adipose-derived adult stem cell, thereby identifying a differentiated adipose-derived adult stem cell.

In one aspect of the present invention, the adipose-derived adult stem cell is a human adipose-derived adult stem cell.

In another aspect of the present invention, the protein is selected from the group consisting of a metabolism-related protein, a heat shock protein, a redox protein, a cytoskeletal protein, a serine protease inhibitor protein, and a protein degradation-related protein.

In yet another aspect of the present invention, the proteomic profile comprises at least two proteins specific for said differentiated adipose-derived adult stem cell.

In still another aspect of the present invention, the protein is selected from the group consisting of stathmin, elfin, LIM, and SH3 domain protein 1.

The present invention comprises a method of distinguishing an undifferentiated adipose-derived adult stem cell from an differentiated adipose-derived adult stem cell, said method comprising comparing a proteomic profile of said undifferentiated adipose-derived adult stem cell to a proteomic profile of a differentiated adipose-derived adult stem cell, wherein the proteomic profile of the differentiated adipose-derived adult stem cell comprises a protein that is specific for the differentiated adipose-derived adult stem cell, further wherein the undifferentiated adipose-derived adult stem cell does not detectably express the protein that is specific for the differentiated adipose-derived adult stem, thereby distinguishing an undifferentiated adipose-derived adult stem cell from a differentiated adipose-derived adult stem cell.

In one aspect of the present invention, the adipose-derived adult stem cell is a human adipose-derived adult stem cell.

In another aspect of the present invention, the protein is selected from the group consisting of a metabolism-related protein, a heat shock protein, a redox protein, a cytoskeletal protein, a serine protease inhibitor protein, and a protein degradation-related protein.

In yet another aspect of the present invention, the proteomic profile comprises at least two proteins specific for said differentiated adipose-derived adult stem cell.

In still another aspect of the present invention, the protein specific for said differentiated adipose-derived adult stem cell is upregulated about 2-fold compared to said undifferentiated adipose-derived adult stem cell.

In another aspect of the present invention, the protein is selected from the group consisting of fatty acid binding protein-adipocyte, heat shock protein 20-like protein, heat shock protein β, heat shock protein 20, heat shock protein 27, heat shock protein 60, plasminogen activator inhibitor-1, pigmented epidermal derived factor, placental thrombin inhibitor, pregnancy zone protein, and protease C1 inhibitor.

The present invention comprises a method of selecting an adipose-derived adult stem cell from a population of adipose-derived adult stem cells, the method comprising comparing a proteomic profile of the adipose-derived adult stem cell to a proteomic profile of the population of adipose-derived adult stem cells, wherein the proteomic profile of the adipose-derived adult stem cell comprises a protein that is specific for the adipose-derived adult stem cell and is not upregulated in the proteomic profile of the population of adipose-derived adult stem cells, thereby selecting an adipose-derived adult stem cell from a population of adipose-derived adult stem cells.

In one aspect of the present invention, the adipose-derived adult stem cell is a human adipose-derived adult stem cell.

In another aspect of the present invention, the protein is selected from the group consisting of a metabolism-related protein, a heat shock protein, a redox protein, a cytoskeletal protein, a serine protease inhibitor protein, and a protein degradation-related protein.

In yet another aspect of the present invention, the proteomic profile comprises at least two proteins specific for said adipose-derived adult stem cell.

In still another aspect of the present invention, the protein specific for said adipose-derived adult stem cell is upregulated about 2-fold compared to said population of adipose-derived adult stem cells.

In another aspect of the present invention, the protein is selected from the group consisting of fatty acid binding protein-adipocyte, heat shock protein 20-like protein, heat shock protein β, heat shock protein 20, heat shock protein 27, heat shock protein 60, plasminogen activator inhibitor-1, pigmented epidermal derived factor, placental thrombin inhibitor, pregnancy zone protein, and protease C1 inhibitor.

The present invention comprises a method of identifying a compound that differentiates an adipose-derived adult stem cell, the method comprising contacting the adipose-derived adult stem cell with the compound, comparing a proteomic profile of the adipose-derived adult stem cell so contacted to a proteomic profile of an adipose-derived adult stem cell not contacted with the compound, wherein the proteomic profile of the adipose-derived adult stem cell so contacted comprises a protein that is specific for a differentiated adipose-derived adult stem cell and is not upregulated in the adipose-derived adult stem cell not contacted with the compound, thereby identifying a compound that differentiates an adipose-derived adult stem cell.

In one aspect of the present invention, the adipose-derived adult stem cell is a human adipose-derived adult stem cell.

In another aspect of the present invention, the protein is selected from the group consisting of a metabolism-related protein, a heat shock protein, a redox protein, a cytoskeletal protein, a serine protease inhibitor protein, and a protein degradation-related protein.

In still another aspect of the present invention, the proteomic profile comprises at least two proteins specific for said differentiated adipose-derived adult stem cell.

In another aspect of the present invention, the protein specific for said differentiated adipose-derived adult stem cell is upregulated.

In yet another aspect of the present invention, the protein is selected from the group consisting of fatty acid binding protein-adipocyte, heat shock protein 20-like protein, heat shock protein β, heat shock protein 20, heat shock protein 27, heat shock protein 60, plasminogen activator inhibitor-1, pigmented epidermal derived factor, placental thrombin inhibitor, pregnancy zone protein, and protease C1 inhibitor.

The present invention further encompasses a compound identified by the method disclosed herein.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1C, is a series of images depicting adipogenesis in human adipose-derived adult stem cells. FIG. 1A depicts Passage 1 human adipose-derived adult stem cells under control (undifferentiated) or differentiated conditions after staining with Oil Red O at 10× magnification. FIG. 1B depicts the same cells with no magnification. FIG. 1C is a graph illustrating the percentage of Oil Red O surface area staining for an entire plate.

FIG. 2, comprising FIGS. 2A through 2C, is a series of images depicting two-dimensional polyacrylamide gel electrophoresis of protein lysates prepared from human adipose-derived adult stem cells in the undifferentiated (FIG. 2B) and differentiated (FIG. 2C) condition following induction. FIG. 2A is an image of a composite gel prepared based on features conserved on replicate gels prepared from protein extracts obtained from individual donors.

FIG. 3, comprising FIGS. 3A and 3B, is a series of graphs depicting the functionality and subcellular localization of identified proteins in undifferentiated human adipose-derived adult stem cells. The subcellular localization (FIG. 3A) and functionality (FIG. 3B) of the protein categories detailed graphically as percentages are based on n=175 individual proteins identified in the undifferentiated adipocyte cells. Abbreviations: C, Cytoplasm; CaBP, Calcium Binding Protein; Chap, Chaperone; Cytoskel, Cytoskeleton; ECM, Extracellular matrix; ER, Endoplasmic Reticulum; GBP, Guanine nucleoside Binding Protein; M, Mitochondria; Metab, Metabolism; N, Nucleus; NR, Not Reported; ProtDeg, Protein Degradation; ProtProc, Protein Processing; RNABP, RNA Binding Protein. The “Other” Location category includes the Golgi, Lysosome, Plasma Membrane, Ribosome, and Secreted while the “Other” Function category includes Amyloid Binding Protein, Cytokine, Ion Channel, Iron Binding Protein, Signal Transduction, and Transcription.

FIG. 4, comprising FIGS. 4A through 4H, is a series of images depicting protein features from 2-D gels prepared with protein lysates from undifferentiated and differentiated human adipose-derived adult stem cells. FIGS. 4A through 4D represent undifferentiated adipose-derived adult stem cells, FIGS. 4E through 4H represent differentiated adipose-derived adult stem cells. The SSP numbers identify the following protein(s): 3101, Fatty acid binding protein-adipocyte; 7204, Heat shock protein 20-like protein; 3107, Stathmin; 6521, Elfin/PDZ and LIM Domain Protein 1 and LIM and SH3 Domain Protein 1. The arrows indicate the location of the protein features.

FIG. 5, comprising FIGS. 5A and 5B, is a series of images depicting immunoblot analysis of heat shock proteins and chaperones. Protein lysates from the undifferentiated (U) and differentiated (D) adipose-derived adult stem cells obtained from two individual donors were detected using antibodies than bind heat shock proteins and chaperones. The average signal intensity ratio (D/U) of the differentiated to undifferentiated cell lysates is indicated.

FIG. 6 is an image depicting the immunoblot detection of Heat Shock Protein 27 phosphoserine 82. Protein lysates from the undifferentiated (U) and differentiated (D) adipose-derived adult stem cells from four individual donors were detected using antibodies to heat shock protein 27 phosphoserine 82 and all isoforms of heat shock protein 27. The average signal intensity ratio (D/U) of the differentiated to undifferentiated cell lysates is indicated.

FIG. 7 is an image depicting the immunoblot detection of Crystallin alpha phosphoproteins. Protein lysates from the undifferentiated (U) and differentiated (D) adipose-derived adult stem cells from four individual donors were detected using antibodies to crystalline alpha β (heat shock protein beta) phosphoserines 19, 45, and 59. The average signal intensity ratio (D/U) of the differentiated to undifferentiated cell lysates is indicated.

FIG. 8, comprising FIGS. 8A through 8J, is a table depicting the protein features of undifferentiated human adipose-derived adult stem cells.

FIG. 9, comprising FIGS. 9A through 9C, is a table depicting proteins that are upregulated ≧2-fold with adipogenesis in human adipose-derived adult stem cells.

FIG. 10 is a table depicting proteins that are downregulated ≧3-fold with adipogenesis in human adipose-derived adult stem cells.

FIG. 11, comprising FIGS. 11A through 11C, is a table depicting proteins identified in undifferentiated and differentiated human adipose-derived adult stem cells.

FIG. 12, comprising FIGS. 12A through 12D, is a table depicting secreted proteins identified in undifferentiated and differentiated human adipose-derived adult stem cells.

FIG. 13, comprising FIGS. 13A through 13X, is a series of images depicting protein features from two-dimensional gels prepared with protein lysates from undifferentiated and differentiated human adipose-derived adult stem cells. FIGS. 13A through 13H depict undifferentiated, and FIGS. 121 through 13P depict differentiated adipose-derived adult stem cells. The SSP numbers identify the following proteins: 3705, pregnancy zone protein precursor; 3208, adiponectin precursor; 1301, calumenin precursor; 4202, heat shock protein 27 (beta 1); 5301, pigment epithelial derived factor precursor (serpin); 5302, pigment epithelial derived factor; 3203, placental thrombin inhibitor (serpin 6); and 7302, plasminogen activator inhibitor I PAI-1. The arrows indicate the location of the protein features. FIGS. 13P through 13X are a series of bar graphs indicating the relative abundance of the spot on the gels from undifferentiated cells (first four bars) versus differentiated (last four bars) adipose-derived adult stem cells.

FIG. 14, comprising FIGS. 14A through 14C, is a series of images depicting two-dimensional polyacrylamide gel electrophoresis of adipose-derived adult stem cells. FIG. 14B depicts gel electrophoresis results from undifferentiated adipose-derived adult stem cells, FIG. 14C depicts gel electrophoresis results from differentiated adipose-derived adult stem cells, and FIG. 14A depicts a master composite of the gels from the two conditions, prepared based on features conserved on replicate gels prepared from protein extracts obtained from the donors.

FIG. 15, comprising FIGS. 15A through 15E, is a series of graphs depicting quantitative real time PCR results for various secreted proteins from undifferentiated and differentiated adipose-derived adult stem cells. FIG. 15A is a graph depicting quantitative real time PCR results from protease C1 inhibitor normalized to cyclophilin B for undifferentiated and differentiated human adipose-derived adult stem cells from individual donors. Values are the mean±S.D. for triplicate determinations for each donor sample. FIG. 15B is a graph depicting quantitative real time PCR results from plasminogen activator inhibitor-1 (PAI-1) normalized to cyclophilin B for undifferentiated and differentiated human adipose-derived adult stem cells from individual donors. Values are the mean±S.D. for triplicate determinations for each donor sample. FIG. 15C is a graph depicting quantitative real time PCR results from pigmented epithelial derived factor (PEDF) normalized to cyclophilin B for undifferentiated and differentiated human adipose-derived adult stem cells from individual donors. Values are the mean±S.D. for triplicate determinations for each donor sample. FIG. 15D is a graph depicting quantitative real time PCR results from crystallin αB normalized to cyclophilin B for undifferentiated and differentiated human adipose-derived adult stem cells from individual donors. Values are the mean±S.D. for triplicate determinations for each donor sample. FIG. 15E is a graph depicting quantitative real time PCR results from heat shock protein 27 normalized to cyclophilin B for undifferentiated and differentiated human adipose-derived adult stem cells from individual donors. Values are the mean±S.D. for triplicate determinations for each donor sample.

### DETAILED DESCRIPTION OF THE INVENTION

The present invention encompasses methods for identifying differentiated mammalian adipose-derived adult stem cells and methods for distinguishing between differentiated and undifferentiated mammalian adipose-derived adult stem cells. Preferably, the mammalian adipose-derived adult stem cells are human adipose-derived adult stem cells. The present invention further comprises methods for distinguishing between different populations of mammalian adipose-derived adult stem cells, whether differentiated or undifferentiated. Preferably the mammalian adipose-derived adult stem cells are human.

Adipogenesis plays a critical role in energy metabolism and is a contributing factor to the obesity epidemic. Further, adipose-derived adult stem cells have vast potential in transplantation, the treatment of degenerative and debilitating diseases, and other therapeutic uses. The present invention is based, in part, on an examination of the proteomic profile of primary cultures of human adipose-derived adult stem cells as a model of adipogenesis. As disclosed elsewhere herein, protein lysates obtained from individual donors of different or the same gender were compared before and after adipocyte differentiation by 2-dimensional gel electrophoresis and tandem mass spectroscopy. Over 170 individual protein features in the undifferentiated adipocyte cells were identified. Following adipogenesis, over 40 proteins were upregulated by ≧2-fold while 13 exhibited a ≧3-fold reduction and/or were downregulated. The majority of the modulated proteins belonged to the following functional categories: cytoskeleton, metabolic, redox, protein degradation, and heat shock protein/chaperones. Additional immunoblot analysis documented the induction of four individual heat shock proteins and confirmed the presence of the heat shock protein 27 phosphoserine 82 isoform, as predicted by the proteomic analysis, as well as the crystallin α phosphorylated isoforms. Thus, the present invention discloses specific proteins that are modulated during adipogenesis, which is useful in identifying pathways and mechanisms related to obesity and type 2 diabetes, as well as providing a novel method of identifying and distinguishing differentiated adipose-derived adult stem cells from undifferentiated adipose-derived cells, as well as method of distinguishing between different populations of adipose-derived adult stem cells.

The present invention is also based, in part, on the discovery that there are shared and distinct proteomic features between undifferentiated and differentiated human adipose-derived adult stem cells isolated from both female and male donors. Further, since human adipose-derived adult stem cells are known to differentiate into chondrocytic, osteoblastic, and neuronal phenotypes under appropriate culture conditions, the ability to distinguish these cells from diverse populations of undifferentiated or differentiated adipose-derived adult stem cells is an important step in using adipose-derived stem cells in therapeutic and diagnostic uses. As an example, the adipose-derived stem cells disclosed herein are known to resemble stromal cells isolated from the bone marrow at the morphologic and differentiation levels. This fact is indicated in the data disclosed herein, which demonstrates homology at the proteome level of greater than half when the present adipose-derived adult stem cells are compared to the proteomic profile of human bone marrow stromal cells, as well as dermal- and synovial-derived fibroblasts.

### Definitions

The present abbreviations are used throughout this application.

ADAS, Adipose Derived Adult Stem; AmyBP, Amyloid Binding Protein; BMI, Body Mass Index; BP, Binding Protein; 2D-PAGE, 2 Dimensional Polyacrylamide Gel Electrophoresis; C, Cytoplasm; C1 inh, Protease C1 Inhibitor; CaBP, Calcium Binding Protein; CarbBP, Carbohydrate Binding Protein; CD, Cluster of Differentiation; Chap, Chaperone/Heat Shock Protein; CM, Conditioned Medium; Cytoskel, Cytoskeleton; D, Differentiated; DMEM, Dulbecco's Modified Eagles Medium; E, Endosome; ECM, Extracellular Matrix; ER, Endoplasmic Reticulum; FeBP, Iron Binding Protein; GBP, GTP Binding Protein; G, Golgi; HSP, Heat Shock Protein; hu, human; Ion Ch, Ion Channel; L, Lysosome; M, Mitochondria; MALDI, Matrix Assisted Laser Desorption/Ionization; Metab, Metabolism; MF, Membrane Fusion; MS, Mass Spectroscopy; mu, murine; N, Nuclear; NR, Not Reported; P, Passage; PAI-1, Plasminogen Activator Inhibitor 1; PEDF, Pigmented Epidermal Derived Factor; PBS, Phosphate Buffered Saline; PM, Plasma Membrane; ProtDeg, Protein Degradation; ProtProc, Protein Processing; Q, Quadrapole; R, Ribosome; Redox, Oxidative-Reduction; S, Secreted; Serpin, Serine Protease Inhibitor; SVF, Stromal Vascular Fraction; Syn, Synthesis; Trans, Transcription; TOF, Time of Flight; U, Undifferentiated; Vaspin, Visceral Adipose tissue-derived Serine Protease Inhibitor

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “adipose tissue-derived cell” refers to a cell that originates from adipose tissue. The initial cell population isolated from adipose tissue is a heterogeneous cell population including, but not limited to stromal vascular fraction (SVF) cells.

“Adipose” refers to any fat tissue. The adipose tissue may be brown or white adipose tissue. Preferably, the adipose tissue is subcutaneous white adipose tissue. The adipose tissue may be from any organism having fat tissue. Preferably the adipose tissue is mammalian, most preferably the adipose tissue is human. A convenient source of human adipose tissue is that derived from liposuction surgery. However, the source of adipose tissue or the method of isolation of adipose tissue is not critical to the invention.

As used herein, the term “adipose-derived adult stem cell (ADAS)” refers to stromal cells that originate from adipose tissue which can serve as stem cell-like precursors to a variety of different cell types such as but not limited to adipocytes, osteocytes, chondrocytes, muscle and neuronal/glial cell lineages. adipose-derived adult stem cells make up a subset population derived from adipose tissue which can be separated from other components of the adipose tissue using standard culturing procedures or other methods disclosed herein. In addition, adipose-derived adult stem cells can be isolated from a mixture of cells using the cell surface markers disclosed herein.

As used herein, the term “adipose cell” is used to refer to any type of adipose tissue, including an undifferentiated adipose-derived adult stem cell and a differentiated adipose-derived adult stem cell.

By the term “applicator,” as the term is used herein, is meant any device including, but not limited to, a hypodermic syringe, a pipette, and the like, for administering the compounds and compositions of the invention.

The term “cytoskeletal protein” is used herein to refer to a protein that provides shape, internal spatial organization, and motility to a cell. Cytoskeletal proteins included, but are not limited to, actin, cofilin 2, destrin (actin-depolymerizing factor), elfin, myosin light chain alkali, transgelin, elfin, lamin A, stathmin, transgelin 2, tropomyosin 1 α chain, and tropomyosin 3 α chain.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation-associated proteins in that cell. For example expression of myelin proteins and formation of a myelin sheath in a glial cell is a typical example of a terminally differentiated glial cell.

When a cell is said to be “differentiating,” as that term is used herein, the cell is in the process of being differentiated.

A “differentiated adipose-derived adult stem cell” is an adipose-derived adult stem cell isolated from any adipose tissue that has differentiated as defined herein.

An “undifferentiated adipose-derived adult stem cell” is a cell isolated from adipose tissue and cultured to promote proliferation, but has no detectably expressed proteins or other phenotypic characteristics indicative of biological specialization and/or adaptation.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulated” is used herein to refer to a decreased amount of expression of a protein in a cell in comparison to another cell capable of encoding the same protein, or decreasing the amount of a protein expressed after the administration of a stimulus, such as a compound, cell culturing conditions, and the like.

“Instructional material,” as that term is used herein, includes a publication, a recording, a diagram, or any other medium of expression which can be used to communicate the usefulness of the composition and/or compound of the invention in the kit for effecting alleviating or treating the various diseases or disorders recited herein. Optionally, or alternately, the instructional material may describe one or more methods of alleviating the diseases or disorders in a cell or a tissue or a mammal, including as disclosed elsewhere herein.

The instructional material of a kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively.

An “isolated cell” refers to a cell which has been separated from other components and/or cells which naturally accompany the isolated cell in a tissue or mammal.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

As applied to a protein, a “fragment” of a polypeptide, protein or an antigen, is about 6 amino acids in length. More preferably, the fragment of a protein is about 8 amino acids, even more preferably, at least about 10, yet more preferably, at least about 15, even more preferably, at least about 20, yet more preferably, at least about 30, even more preferably, about 40, and more preferably, at least about 50, more preferably, at least about 60, yet more preferably, at least about 70, even more preferably, at least about 80, and more preferably, at least about 100 amino acids in length amino acids in length.

A “genomic DNA” is a DNA strand which has a nucleotide sequence homologous with a gene as it exists in the natural host. By way of example, a fragment of a chromosome is a genomic DNA.

A “heat shock protein” is used herein to refer to a protein that functions in response to hyperthermia and other environmental stresses to increase thermal tolerance and perform functions essential to cell survival under these conditions. Heat shock proteins include, but are not limited to, Cyclophilin A, HSPβ6 (crystallin), HSP 20-like protein, Cyclophilin B, FK Binding protein 2, and HSP27.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are completely or 100% homologous at that position. The percent homology between two sequences is a direct function of the number of matching or homologous positions, e.g., if half (e.g., five positions in a polymer ten subunits in length) of the positions in two compound sequences are homologous then the two sequences are 50% identical, if 90% of the positions, e.g., 9 of 10, are matched or homologous, the two sequences share 90% homology. By way of example, the DNA sequences 5′ ATTGCC3′ and 5′TATGGC3′ share 50% homology.

In addition, when the terms “homology” or “identity” are used herein to refer to the nucleic acids and proteins, it should be construed to be applied to homology or identity at both the nucleic acid and the amino acid sequence levels.

A “metabolism-related protein” is used herein to refer to a protein that is part of the transformation by which energy is made available for the uses of an organism. Metabolism-related proteins include, but are not limited to, apolipoprotein A-1, ATP synthase A chain, carbonic anhydrase II, electron transfer flavoprotein alpha-subunit, enoyl CoA hydratase, fatty acid binding protein (adipocyte), fumarylacetoacetase, glyceraledehyde-3-phosphate dehydrogenase, glycerol-3-phosphate dehydrogenase [NAD+], cytoplasmic, isocitrate dehydrogenase, phosphoglycerate kinase 1, pyruvate dehydrogenase D-3, 2-oxisovalerate dehydrogenase α subunit, succinyl CoA ketoacid coenzyme A transferase 1, glyceraldehyde 3-phosphate, dehydrogenase, and enoyl CoA hydratase.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

A “protein degradation-related protein” is used herein to refer to a protein that is involved in the destruction of other proteins within a cell. Protein degradation-related proteins include, but are not limited to, cathepsin B, ubiquitin protein ligase, ubiquitin, phosphatidylethanolamine-binding protein, ras-related protein Rab-6A, and syntaxin 7.

The term “proteomic profile” is used herein to refer the detectable manifestation of the proteins produced from the information encoded by a genome. As an example, the proteomic profile of a cell can comprise a gel, an immunoblot or another means that displays the proteins detected in a cell.

The term “proteome” is used herein to refer to the proteins produced from the information encoded by a genome.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “redox protein”, as used herein, refers to a protein involved in the reduction-oxidation pathway. Redox protein include, but are not limited to, cytochrome b5, cytochrome c oxidase polypeptide Vb, flavin reductase, NRH dehydrogenase, peroxiredoxin 1 (thioredoxin peroxidase 2), peroxiredoxin 2, and superoxide dismutase Cu—Zn.

A “serine protease inhibitor”, as used herein, refers to a protein that inhibits, reversibly or irreversibly, the activity of a serine protease. Serine protease inhibitors include, but are not limited to, plasminogen activator inhibitor 1 (PAI-1), plasminogen activator inhibitor 2 (PAI-2), pigmented epidermal derived factor (PEDF), placental thrombin inhibitor, pregnancy zone protein, protease C1 inhibitor (C1 inh), protease nexin-1, alpha 1-antitrypsin, alpha 1-antichymotrypsin, alpha 2-antiplasmin, antithrombin, complement 1-inhibitor, neuroserpin, and protein Z-related protease inhibitor (ZPI).

By “tag” polypeptide is meant any protein which, when linked by a peptide bond to a protein of interest, may be used to localize the protein, to purify it from a cell extract, to immobilize it for use in binding assays, or to otherwise study its biological properties and/or function.

A “therapeutic” treatment is a treatment administered to a patient who exhibits signs of pathology for the purpose of diminishing or eliminating those signs and/or decreasing or diminishing the frequency, duration and intensity of the signs.

By the term “specifically binds,” as used herein, is meant an antibody, or a ligand, which recognizes and binds with a cognate binding partner present in a sample, but which antibody or ligand does not substantially recognize or bind other molecules in the sample.

To “treat” a disease as the term is used herein, means to reduce the frequency of the disease or disorder reducing the frequency with which a symptom of the one or more symptoms disease or disorder is experienced by an animal.

The term “upregulated” is used herein to refer to an increased amount of expression of a protein in a cell in comparison to another cell capable of encoding the same protein, or increasing the amount of a protein expressed after the administration of a stimulus, such as a compound, cell culturing conditions, and the like.

### Description

The invention relates to the discovery that certain proteins are differentially expressed on differentiated versus undifferentiated adipose-derived adult stem cells. Methods of identifying such proteins allow the identification of a differentiated adipose-derived adult stem cell. The identification of such proteins also allows one to distinguish between undifferentiated and differentiated adipose-derived adult stem cells. The present invention also facilitates the identification of a homogenous population of adipose-derived adult stem cells in a population of heterogeneous differentiated, undifferentiated, or a mixed population of adipose or adipose-derived cells.

### I. Methods

The invention includes a method of identifying an adipose-derived adult stem cell. The method comprises identifying proteins expressed by a cell and determining if the proteins expressed by the cell are proteins that are, as disclosed herein, specific for differentiated adipose-derived adult stem cells. That is, the present method comprises identifying an adipose-derived adult stem cell by comparing the proteomic profile of an adipose-derived adult stem cell to the proteomic profile of another adipose-derived adult stem cell or to a known proteome, such as those disclosed herein. The method further comprises identifying a protein in the proteomic profile of the adipose-derived adult stem cell that is specific for an adipose-derived adult stem cell and is not upregulated in the proteomic profile of another adipose-derived adult stem cell. Methods for isolating an adipose-derived adult stem cell from adipose tissue are known in the art and are described elsewhere herein. The classes of proteins identified by the present methods include, without limitation, metabolism-related proteins, heat shock/chaperone-related proteins, proteins involved in reduction/oxidation (redox), cytoskeletal-related proteins, proteins related to protein degradation and processing, serine protease inhibitors (serpins), and other classes and types of proteins disclosed elsewhere herein. Proteins that are upregulated during differentiation and/or adipogenesis of a human adipocyte-derived adult stem cell, and are therefore proteins specific for an adipose-derived adult stem cell include, but are not limited to, apolipoprotein A-1, ATP synthase Δ chain, carbonic anhydrase II, electron transfer flavoprotein alpha-subunit, enoyl CoA hydratase, fatty acid binding protein (adipocyte), fumarylacetoacetase, glyceraledehyde-3-phosphate dehydrogenase, glycerol-3-phosphate dehydrogenase [NAD+], cytoplasmic, isocitrate dehydrogenase, phosphoglycerate kinase 1, pyruvate dehydrogenase D-3, 2-oxisovalerate dehydrogenase α subunit, succinyl CoA ketoacid coenzyme A transferase 1, cyclophilin A, HSPβ6 (crystallin), HSP 20-like protein, HSP27, cytochrome b5, cytochrome c oxidase polypeptide Vb, flavin reductase, NRH dehydrogenase, peroxiredoxin 1 (thioredoxin peroxidase 2), peroxiredoxin 2, superoxide dismutase Cu—Zn, actin, cofilin 2, destrin (Actin-depolymerizing factor), elfin, myosin light chain alkali, transgelin, cathepsin B, ubiquitin protein ligase, phosphatidylethanolamine-binding protein, ras-related protein rab-6A, syntaxin 7 ectodysplasin A, G2 and S phase expressed protein, kinesin 2, LIM and SH3 domain protein 1, low affinity Ig Fc ε receptor, phosphatidyl inositol 4 kinase α, plasma retinal binding protein, S100 calcium-binding protein A13, SH3 domain binding glutamic rich protein, STRAIT11499, transgelin 2, PAI-1, PEDF, placental thrombin inhibitor, pregnancy zone protein, protease C1 inhibitor (C1 inh), and UMP-CMP kinase.

Preferably, the method of the present invention comprises identifying an upregulated protein that is specific for an adipose-derived adult stem cell where the protein is upregulated at least about 2-fold compared to another adipose-derived adult stem cell. A protein upregulated at least about 2-fold comprises a detectable increase in the expression of a protein that is upregulated about 2-fold greater than before differentiation of the cell, or is about 2-fold in comparison to an undifferentiated adipose-derived adult stem cell. Even more preferably, the method of the present invention comprises identifying a detectably upregulated amount of enoyl CoA hydratase, fumarylacetoacetase, phosphoglycerate kinase 1, 2-Oxisovalerate dehydrogenase α subunit, cyclophilin A, flavin reductase, NRH dehydrogenase, actin, cofilin 2, destrin (actin-depolymerizing factor), myosin light chain alkali, transgelin, ubiquitin protein ligase, ras-related protein rab-6A, ectodysplasin A, G2 and S phase expressed protein, kinesin 2, LIM and SH3 domain protein 1, low affinity Ig Fc ε receptor, phosphatidyl inositol 4 kinase α, plasma retinal binding protein, SH3 domain binding glutamic rich protein, STRAIT11499, transgelin 2, PAI-1, PEDF, placental thrombin inhibitor, pregnancy zone protein, protease C1 inhibitor (C1 inh), and UMP-CMP kinase. The method of the present invention comprises identifying an adipose-derived adult stem cell by detecting the expression of these proteins on an adipose-derived adult stem cell. Methods for detecting particular proteins disclosed herein in order to identify an adipose-derived adult stem cell are disclosed elsewhere herein.

The present invention further comprises a method of identifying an adipose-derived adult stem cell by identifying proteins where expression of the protein(s) is reduced and/or downregulated during differentiation of an adipose-derived adult stem cell, when compared to an undifferentiated adipose-derived adult stem cell. Thus, the present invention comprises identifying an adipose-derived adult stem cell by comparing the proteomic profile of an adipose-derived adult stem cell to the proteomic profile of an another adipose-derived adult stem cell. This is because, as demonstrated elsewhere herein, differentiated adipose-derived adult stem cells can be identified by the reduced and/or downregulated expression of various proteins when compared to the proteomic profile of an undifferentiated adipose-derived adult stem cell in which the proteins are not down regulated. These reduced and/or downregulated proteins include, without limitation, glyceraldehyde 3-phosphate, dehydrogenase, enoyl CoA hydratase, cyclophilin B, FK binding protein 2, HSP27, elfin, lamin A, stathmin, transgelin 2, tropomyosin 1 α chain, tropomyosin 3 α chain, annexin 2, hnRNP A2/B1, translocon associated protein δ, platelet activating factor acetylhydrolase 1B. Preferably, the method of the present invention comprises identifying an reduction of at least about greater than 3-fold of these proteins. Even more preferably, the method of the present invention comprises identifying the reduction of enoyl CoA hydratase, elfin, stathmin, transgelin 2, translocon associated protein δ, platelet activating factor acetylhydrolase 1B.

The present invention further comprises a method of distinguishing an undifferentiated adipose-derived adult stem cell from a differentiated adipose-derived adult stem cell. The present method comprises comparing the proteomic profile of a cell, such as undifferentiated adipose-derived adult stem, to the proteomic profile of another cell, such as a differentiated adipose-derived adult stem cell, where the proteomic profile of the differentiated adipose-derived adult stem cell comprises a protein that is specific for a differentiated adipose-derived adult stem cell, and the undifferentiated adipose-derived adult stem cell does not detectably express the protein, thereby distinguishing an undifferentiated adipose-derived adult stem cell from a differentiated adipose-derived adult stem cell. This is because, as demonstrated by the data disclosed herein, the proteomic profile of an adipose-derived adult stem cell can be used to distinguish between an undifferentiated adipose-derived adult stem cell and a differentiated adipose-derived adult stem cell. Thus, the present invention provides a method of identifying a cell that can be used to illuminate the processes leading to type 2 diabetes and obesity. The present invention further provides a method for identifying a multipotent adipose-derived adult stem cell for the use in, inter alia, therapy of various degenerative and other diseases.

An undifferentiated adipose-derived adult stem cell is distinguished from a differentiated adipose-derived adult stem cell using the methods disclosed elsewhere herein. Specifically, the proteomic profile of one type of cell, for example an undifferentiated adipose-derived adult stem cell, is compared to the proteomic profile of another cell, for example a differentiated adipose-derived adult stem cell. Comparison of the proteomic profile of the first cell and the second cell, and the different protein expression profiles in the two cells results in a method of distinguishing the cells from each other, despite morphological or other similarities.

The present invention further comprises a method of selecting different populations of differentiated adipose-derived adult stem cells. That is, the present invention comprises a method of differentiating between different types of cells within a larger population of differentiated adipose-derived adult stem cells. The present invention comprises comparing the proteomic profile of a first population of differentiated adipose-derived adult stem cells to the proteomic profile of a second population of differentiated adipose-derived adult stem cells and identifying a protein that is specific for a population of differentiated adipose-derived adult stem cell and is not upregulated in the second population of differentiated adipose-derived adult stem cells. A population of differentiated adipose-derived adult stem cells is selected from a larger population of differentiated adipose-derived adult stem cells using the methods disclosed elsewhere here. In particular, the proteomic profile of one population of cells, for example differentiated adipose-derived adult stem cells, is compared to the proteomic profile of another population of cells, for example a larger population of differentiated adipose-derived adult stem cells. Comparison of the proteomic profile of the smaller population to the larger population, and the different protein expression profiles in the two cell populations results in a method of selecting the desired population of cells.

The methods of the present invention can further be used to identify novel means of differentiating a cell or novel means of specifically differentiating a cell towards a desired lineage. That is, according to the methods of the present invention, a cell or population of cells can be treated or otherwise contacted with a putative differentiating compound in order to determine if the compound initiates differentiation of an adipose-derived adult stem cell. Once the adipose-derived adult stem cell has been contacted with such an agent, the proteomic profile of the cell can be analyzed according to the methods of the present invention in order to determine if the compound is capable of causing differentiation of an adipose-derived adult stem cell. The present method further comprises identifying a protein that is specific for the adipose-derived adult stem cell contacted with the compound, but is not upregulated in the adipose-derived adult stem cell not contacted with the compound. The criteria for determining if a cell is differentiated, using the methods of the present invention, are disclosed elsewhere herein. In addition, in order to determine if a differentiating compound directs a cell towards a certain desired lineage, a cell or population of cells can be treated or otherwise contacted with a putative differentiating compound in order to determine if the compound initiates differentiation of an adipose-derived adult stem cell towards a desired lineage. Once the adipose-derived adult stem cell has been contacted with such an compound, the proteomic profile of the cell can be analyzed according to the methods of the present invention in order to determine if the compound is capable of causing differentiation of an adipose-derived adult stem cell towards a desired lineage. Methods for determining if a cell is differentiated are disclosed elsewhere herein.

Alternatively, the proteomic profile of a cell or a population of cells can be compared with the proteomic profile of a reference sample of an undifferentiated or differentiated adipose-derived adult stem cell, such as those provided herein. Thus, the differentiation state of an adipose-derived adult stem cell can be determined from a cell or a population of cells by reference to a standard.

The present invention concerns methods for differentiating, distinguishing and identifying an adipose-derived adult stem cell based upon the proteomic profile of a cell. The invention employs proteomics techniques well known in the art, as described, for example, in the following textbooks, the contents of which are hereby incorporated by reference: Proteome Research: New Frontiers in Functional Genomics (Principles and Practice), M. R. Wilkins et al., eds., Springer Verlag, 1007; 2-D Proteome Analysis Protocols, Andrew L Link, editor, Humana Press, 1999; Proteome Research: Two-Dimensional Gel Electrophoresis and Identification Methods (Principles and Practice), T. Rabilloud editor, Springer Verlag, 2000; Proteome Research: Mass Spectrometry (Principles and Practice), P. James editor, Springer Verlag, 2001; Introduction to Proteomics, D. C. Liebler editor, Humana Press, 2002; Proteomics in Practice: A Laboratory Manual of Proteome Analysis, R. Westermeier et al., eds., John Wiley & Sons, 2002.

Methods for isolating an adipose-derived adult stem cell from adipose tissue, including, for example liposuction aspirates, biological samples comprising adipose tissue, cultured adipose tissue, and the like, are known in the art. Such methods are disclosed in, for example, U.S. Pat. Nos. 6,777,231, 6,569,633, 6,555,374, 6,492,130, 6,429,013, and 6,391,297, all of which are incorporated by reference herein.

One skilled in the art will recognize many methods and materials similar or equivalent to those described herein, which could be used in the practice of the present invention. Indeed, the present invention is in no way limited to the methods and materials described.

According to the present invention, proteomics analysis of biological samples, tissues, cell cultures, and the like can be performed using a variety of methods known in the art. Typically, protein patterns (proteome maps) of samples from different sources, such as a differentiated adipose-derived adult stem cell or an undifferentiated adipose-derived adult stem cell sample, are compared to detect proteins that are up- or down-regulated in a morphogenic, developmental or differentiated state of the cell or sample. These proteins can then be excised for identification and full characterization, e.g. using peptide-mass fingerprinting and/or mass spectrometry and sequencing methods, or the undifferentiated and/or differentiated state-specific proteomic profile can be used directly for the diagnosis of the state of differentiation of interest, or to confirm the presence or absence of differentiation.

The required amount of total proteins in the samples will depend on the analytical technique used, and can be readily determined by one skilled in the art according to the methods disclosed herein. The proteins present in the cellular samples are typically separated by two-dimensional gel electrophoresis according to their pI and molecular weight. The proteins are first separated by their charge using isoelectric focusing (one-dimensional gel electrophoresis). This step can, for example, be carried out using immobilized pH-gradient (IPG) strips, which are commercially available. The second dimension is an SDS-PAGE analysis, where the focused IPG strip is used as the sample. After 2-D gel separation, proteins are visualized with conventional dyes, like Coomassie Blue, Sypro Ruby or silver staining, and imaged using known techniques and equipment, such as, e.g. Bio-Rad GS800 densitometer and PDQUEST software, both of which are commercially available. Individual spots are then cut from the gel, destained, and subjected to tryptic digestion. The peptide mixtures can be analyzed by mass spectrometry (MS) or other means disclosed elsewhere herein. Alternatively, the peptides can be separated, for example by capillary high performance liquid chromatography (HPLC) and can be analyzed by MS either individually, or in pools.

The present invention is not limited to the use of 2D gel electrophoresis for distinguishing and/or identifying undifferentiated adipose-derived adult stem cells or differentiated adipose-derived adult stem cells. Gel electrophoresis techniques are well known to one of ordinary skill in the art. Electrophoresis is the process of separating molecules on the basis of the molecule's migration through a gel in an applied electric field. In an electric field, a molecule will migrate towards the pole (cathode or anode) that carries a charge opposite to the net charge carried by the molecule. This net charge depends in part on the pH of the medium in which the molecule is migrating. One common electrophoretic procedure is to establish solutions having different pH values at each end of an electric field, with a gradient range of pH in between. At a certain pH, the isoelectric point of a molecule is obtained and the molecule carries no net charge. As the molecule crosses the pH gradient, it reaches an isoelectric point and is thereafter immobile in the electric field. Therefore, this electrophoresis procedure separates molecules according to their different isoelectric points.

Electrophoresis in a polymeric gel, such as a polyacrylamide gel or an agarose gel, adds two advantages to an electrophoretic system. First, the polymeric gel stabilizes the electrophoretic system against convective disturbances. Second, the polymeric gel provides a porous passageway through which the molecules must travel. Since larger molecules will travel more slowly through the passageways than smaller molecules, use of a polymeric gel permits the separation of molecules by both molecular size and isoelectric point.

Molecules with different isoelectric points, such as proteins, can be denatured in a solution of detergent, such as sodium dodecyl sulfate (SDS). The SDS-covered proteins have similar isoelectric points and therefore migrate through the gel on the basis of molecular size.

The present invention encompasses the use of high-resolution electrophoresis, e.g., one or two-dimensional gel electrophoresis to separate proteins from a cell or a population of cells. Preferably, two-dimensional gel electrophoresis is used to generate two-dimensional array of spots of proteins from a sample, which may indicate those proteins involve in stem cell transplantation.

Two-dimensional gel electrophoresis can be performed using methods described herein and in, for example, U.S. Pat. Nos. 5,534,121 and 6,398,933. Typically, proteins in a sample are separated by, e.g., isoelectric focusing, during which proteins in a sample are separated in a pH gradient until they reach a spot where their net charge is zero (i.e., isoelectric point). This first separation step results in one-dimensional array of proteins. The proteins in one dimensional array are further separated using a technique generally distinct from that used in the first separation step. For example, in the second dimension, proteins separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of the protein. Typically, two-dimensional gel electrophoresis can separate chemically different proteins in the molecular mass range from 1000-200,000 Da within complex mixtures.

The present invention further encompasses the use of isoelectrofocusing to identifying adipose-derived adult stem cell derived proteins, thereby identifying, differentiating and/or distinguishing undifferentiated adipose-derived adult stem cells from differentiated adipose-derived adult stem cells. Using this technique, proteins are extracted from cells using a lysis buffer. To facilitate an efficient process, this lysis buffer should be compatible with that of additional separation and analysis steps to be employed (e.g., reverse-phase, HPLC and mass spectrometry) in order to allow direct use of the products from each step into subsequent steps. Such a buffer is an important aspect of automating the process. Thus, the preferred buffer should meet two criteria: it solubilizes proteins and it is compatible with each of the steps in the separation/analysis methods. One skilled in the art can determine the suitability of a buffer for any particular configuration by solubilizing a protein sample in the buffer. If the buffer solubilizes the protein, the sample is run through the particular configuration of separation and detection methods desired. A positive result is achieved if the final step of the desired configuration produces detectable information (e.g., ions are detected in a mass spectrometry analysis). Alternately, the product of each step in the method can be analyzed to determine the presence of the desired product (e.g., determining whether protein elutes from the separation steps).

After extraction in the lysis buffer, proteins are initially separated in a first dimension. The proteins are isolated in a liquid fraction that is compatible with subsequent techniques (reverse phase HPLC) and mass spectrometry steps. n-octyl β-D-glucopyranoside (OGI, from Sigma) may be used in the buffer. This is one of the few detergents that are compatible with both reverse-phase chromatography and HPLC and subsequent mass spectrometry analyses.

After extraction, the supernatant protein solution is loaded to a device that can separate the proteins according to their pI by isoelectric focusing (IEF). The proteins are solubilized in a running buffer that again should be compatible with reverse phase HPLC. A suitable running buffer is 6 M urea, 2 M thiourea, 0.5% n-octyl β-D-glucopyranoside, 10 mM dithioerythritol and 2.5% (w/v) carrier ampholytes (3.5 to 10 pI).

The present invention further comprises the use of various methods for identifying a protein. As disclosed elsewhere herein, mass spectrophotometry can be used to identify an adipose-derived adult stem cell protein, but other methods known in the art and/or described herein can also be used to identify a protein from an adipose-derived adult stem cell.

Mass spectrometers consist of an ion source, mass analyzer, ion detector, and data acquisition unit. First, the peptides are ionized in the ion source. Then the ionized peptides are separated according to their mass-to-charge ratio in the mass analyzer and the separate ions are detected. Mass spectrometry has been widely used in protein analysis, especially since the invention of matrix-assisted laser-desorption ionisation/time-of-flight (MALDI-TOF) and electrospray ionization (ESI) methods. There are several versions of mass analyzer, including, for example, MALDI-TOF and triple or quadrupole-TOF, or ion trap mass analyzer coupled to ESI. Thus, for example, a Q-TOF-2 mass spectrometer uses an orthogonal time-of-flight analyzer that allows the simultaneous detection of ions across the full mass spectrum range. For further details see, e.g. Chemusevich et al., (2001, J. Mass Spectrom. 36: 849-865).

In some embodiments of the present invention, the proteins are characterized using mass spectrometry. For example, the proteins that elute from the chromatography separation are analyzed by mass spectrometry to determine their molecular weight and identity. For this purpose the proteins eluting from the separation can be analyzed simultaneously to determine molecular weight and identity. A fraction of the effluent is used to determine molecular weight by either matrix-assisted laser desorption ionization (MALDI-TOF-MS) or electrospray spectrometry (ESI) or time-of-flight (TOF) (LCT, Micromass) (See e.g., U.S. Pat. No. 6,002,127). The remainder of the eluent can be used to determine the identity of the proteins via digestion of the proteins and analysis of the peptide mass map fingerprints by either MALDI-TOF-MS or ESI or TOF. The molecular weight 2D protein map is matched to the appropriate digest fingerprint by correlating the molecular weight total ion chromatograms with the UV-chromatograms and by calculation of the various delay times involved. The UV-chromatograms are automatically labeled with the digest fingerprint fraction number. The resulting molecular weight and digest mass fingerprint data can then be used to search for the protein identity via web-based programs like MSFit (UCSF).

Separated proteins may be analyzed by mass spectrometry to facilitate the generation of detailed and informative 2D protein maps. The nature of the mass spectrometry technique utilized for analysis in the present invention may include, but is not limited to, ion trap mass spectrometry, ion trap/time-of-flight mass spectrometry, quadrupole and triple quadrupole mass spectrometry, Fourier Transform (ICR) mass spectrometry, and magnetic sector mass spectrometry. Applications of mass spectrometric methods are well-known to those of skill in the art and are discussed in Methods in Enzymology, In: Mass Spectrometry, McCloskey (Ed.), Academic Press, NY, Vol. 193, 1990.

Various MS techniques can be used to further analyze the subfractions for detailed identification and characterization of the proteins. Moreover, the second dimension can run directly to an MS, whereby both the UV/pI maps as well as the mass/pI maps for the intact proteins can be obtained using the software to display both. Having the mass analysis of the intact proteins allows for direct comparison with the matrix-assisted laser desorption ionization (MALDI) peptide mass mapping analysis of the protein to observe differences between the intact molecular weight (MW) and the database MW values.

Mass spectroscopy measures the charge-to-mass ratio of an ionized protein or peptide fragment. Mass spectrometers have been used to identify specific proteins with a known mass extraction from two-dimensional electrophoresis gels. However, because proteins may be too large to be analyzed directly by MS, the protein or spot excised from a gel can be proteolytically digested into smaller peptide fragments. The mass of each of these peptides can be measured in the spectrometer, creating a profile of component peptide masses which, when compared to the known mass of the undigested protein, define a “peptide mass fingerprint” characteristic for a specific protein. A protein can be identified by comparing its peptide mass fingerprints with fingerprints produced by in vitro digestion of every protein in a database.

The present invention further encompasses other forms of spectroscopy and chromatography for the identification, differentiation and/or distinguishing of undifferentiated adipose-derived adult stem cells or differentiated adipose-derived adult stem cells, and the proteins expressed therefrom. Chromatography techniques are well known in the art. These techniques are used to separate organic compounds on the basis of their charge, size, shape, and their solubilities. Chromatography consists of a mobile phase (solvent and the molecules to be separated) and a stationary phase either of paper (in paper chromatography) or glass beads, called resin, (in column chromatography) through which the mobile phase travels. Molecules travel through the stationary phase at different rates because of their chemistry. Types of chromatography that may be employed in the present invention include, but are not limited to, high performance liquid chromatography (HPLC), ion exchange chromatography (IEC), and reverse phase chromatography (RP). Other kinds of chromatography include: adsorption, partition, affinity, gel filtration and molecular sieve, and many specialized techniques for using them including column, paper, thin-layer and gas chromatography (Freifelder, In: Physical Biochemistry Applications to Biochemistry and Molecular Biology, 2nd ed. Wm. Freeman and Co., NY, 1982).

High performance liquid chromatography (HPLC) is similar to reverse phase, only in this method, the process is conducted at a high velocity and pressure drop. The column is shorter and has a small diameter, but it is equivalent to possessing a large number of equilibrium stages.

High-performance chromatofocusing (HPCF) produces liquid pI fractions as the first-dimension of protein separation followed by high-resolution reversed-phase (RP) HPLC of each of the pI fractions as the second dimension. Proteins are mapped (like gels), but the liquid fractions make for easy interface with mass spectrometry (MS) for detailed intact protein characterization and identification (unlike gels) on more selective basis without resorting to protein digestion.

Using HPCF columns, 15-20 total pI fractions-are typically collected over the pH range of 8.5-4.0. Each liquid pI fraction ideally has pI ranges from 0.2 to 0.3 units. These fractions are then analyzed by RP-HPLC to produce high-resolution 2D maps of the expressed proteins present in the sample. Software converts complex chromatograms into easily visualized 2-D maps plotting pI versus retention time (UV signal). These UV pI maps allow for easy comparisons of all intact proteins present in the sample across all the pI fractions. In essence they are pI-hydrophobicity 2D maps.

In some embodiments of the invention, it is contemplated that multi-dimensional protein separation may comprise reversed phase chromatography. Reversed phase chromatography (RPC) utilizes solubility properties of the sample by partitioning it between a hydrophilic and a lipophilic solvent. The partition of the sample components between the two phases depends on their respective solubility characteristics. Less hydrophobic components end up primarily in the hydrophilic phase while more hydrophobic ones are found in the lipophilic phase. In RPC, silica particles covered with chemically-bonded hydrocarbon chains (2-18 carbons) represent the lipophilic phase, while an aqueous mixture of an organic solvent surrounding the particle represents the hydrophilic phase.

When a sample component passes through an RPC column the partitioning mechanism operates continuously. Depending on the extractive power of the eluent, a greater or lesser part of the sample component is retained reversibly by the lipid layer of the particles, in this case called the stationary phase. The larger the fraction retained in the lipid layer, the slower the sample component moves down the column. Hydrophilic compounds move faster than hydrophobic ones, since the mobile phase is more hydrophilic than the stationary phase.

Compounds stick to reverse phase HPLC columns in high aqueous mobile phase and are eluted from RP HPLC columns with high organic mobile phase. In RP HPLC compounds are separated based on their hydrophobic character. Peptides can be separated by running a linear gradient of the organic solvent.

Ion exchange chromatography (IEC) is applicable to the separation of almost any type of charged molecule, from large proteins to small nucleotides and amino acids. It is very frequently used for proteins and peptides, under widely varying conditions. In protein structural work the consecutive use of gel permeation chromatography (GPC) and IEC is quite common.

In ion exchange chromatography, a charged particle (matrix) binds reversibly to sample molecules (proteins, etc.). Desorption is then brought about by increasing the salt concentration or by altering the pH of the mobile phase. Ion exchange containing diethyl aminoethyl (DEAE) or carboxymethyl (CM) groups is most frequently used in biochemistry. The ionic properties of both DEAE and CM are dependent on pH, but both are sufficiently charged to work well as ion exchangers within the pH range 4 to 8 where most protein separations take place.

The property of a protein which governs its adsorption to an ion exchanger is the net surface charge. Since surface charge is the result of weak acidic and basic groups of a protein, separation is highly pH dependent. Going from low to high pH values, the surface charge of proteins shifts from a positive to a negative charge surface charge. The pH versus net surface curve is a individual property of a protein, and constitutes the basis for selectivity in IEC. At a pH value below its isoelectric point a protein (+ surface charge) will adsorb to a cation exchanger (−) such as one containing CM groups. Above the isoelectric point a protein (− surface charge) will adsorb to a anion exchanger (+), e.g., one containing DEAE-groups.

As in all forms of liquid chromatography, conditions are employed that permit the sample components to move through the column with different speeds. At low ionic strengths, all components with affinity for the ion exchanger are tightly adsorbed at the top of the ion exchanger and nothing remains in the mobile phase. When the ionic strength of the mobile phase is increased by adding a neutral salt, the salt ions compete with the protein and more of the sample components are partially desorbed and start moving down the column. Increasing the ionic strength even more causes a larger number of the sample components to be desorbed, and the speed of the movement down the column to increase. The higher the net charge of the protein, the higher the ionic strength needed to bring about desorption. At a certain high level of ionic strength, all the sample components are fully desorbed and move down the column with the same speed as the mobile phase.

Further to spectrometry or chromatography identification, the amino acid sequences of the peptide fragments and eventually the proteins from which they are derived can be determined by techniques known in the art, such as certain variations of mass spectrometry, or Edman degradation.

The method of the present invention comprises using the techniques described herein to identify, distinguish and differentiate between different populations of adipose-derived adult stem cells, including undifferentiated adipose-derived adult stem cells and differentiated adipose-derived adult stem cells. As noted before, in the context of the present invention the term “proteomic profile” is used to refer to a representation of the expression pattern of a plurality of proteins in a biological sample, e.g. a population of cells in varying states of cellular differentiation. The proteomic profile can, for example, be represented as a mass spectrum, but other representations based on any physicochemical or biochemical properties of the proteins are also included in the present invention. Although it is possible to identify and sequence all or some of the proteins present in the proteomic profile of a cell or a population of cells, it is not necessary for the use of the proteomic profiles generated in accordance with the present invention. Identification of a particular differentiation state can be based on characteristic differences (unique expression signatures) between, for example, an undifferentiated proteomic profile, and a differentiated or adipose-derived adult stem cell proteomic profile. The unique expression signature can be any unique feature or motif within the proteomic profile of a cell or population of cells that differs from the proteomic profile of another cell or population of cells. For example, if the proteomic profile is presented in the form of a mass spectrum, the unique expression signature is typically a peak or a combination of peaks that differ, qualitatively or quantitatively, from the mass spectrum of another cell or population of cells. Thus, the appearance of a new peak or protein, or a combination of new peaks or proteins in the mass spectrum, in a gel, in an immunoblot, or in any of the other means described herein or known in the art, or any statistically significant change in the amplitude or shape of an existing peak or combination of existing peaks in the mass spectrum, or the presence or absence of a protein can be considered a unique expression signature. When the proteomic profile of a cell or population of cells is compared with the proteomic profile of another cell or population of cells, the difference after such a comparison is indicative of alternate states of differentiation or different populations of differentiated cells in a larger population.

Statistical methods for comparing proteomic profiles are well known in the art. For example, in the case of a mass spectrum, the proteomic profile is defined by the peak amplitude values at key mass/charge (M/Z) positions along the horizontal axis of the spectrum. Accordingly, a characteristic proteomic profile can, for example, be characterized by the pattern formed by the combination of spectral amplitudes at given M/Z vales. The presence or absence of a characteristic expression signature, or the substantial identity of two profiles can be determined by matching the proteomic profile (pattern) of a cell with the proteomic profile (pattern) of a reference or another cell, with an appropriate algorithm. A statistical method for analyzing proteomic patterns is disclosed, for example, in Petricoin, et al., (2002, The Lancet, 359: 572-77; Issaq et al., 2002, Biochem Biophys Commun, 292: 587-92; Ball et al., 2002, Bioinformatics 18: 395-404; Li, et al., 2002, Clinical Chemistry Journal, 48: 1296-1304).

In addition to the methods disclosed herein for distinguishing, differentiating, or identifying the differentiation state of an adipose-derived adult stem cell, other methods of elucidating the proteomic profile of a cell can be used in the methods of the present invention. As an example, the methods of the present invention can also be performed using protein arrays. Protein arrays have gained wide recognition as a powerful means to detect proteins, monitor protein expression levels, and investigate protein interactions and functions. These arrays enable high-throughput protein analysis, when large numbers of determinations can be performed simultaneously, using automated means. In the microarray or chip format, such determinations can be carried out with minimum use of materials while generating large amounts of data.

Protein arrays are formed by immobilizing proteins on a solid surface, such as glass, silicon, micro-wells, nitrocellulose, PVDF membranes, and microbeads, using a variety of covalent and non-covalent attachment chemistries known in the art. Preferably, the solid support is chemically stable before and after the coupling procedure, allows good spot morphology, displays minimal nonspecific binding, does not contribute a background in detection systems, and is compatible with different detection systems.

In general, protein microarrays use the same detection methods commonly used for the reading of DNA arrays. Similarly, the same instrumentation as used for reading DNA microarrays is applicable to protein arrays.

As an example, capture arrays (e.g. antibody arrays) can be probed with fluorescently labeled proteins from two different sources, such as differentiated and undifferentiated adipose-derived adult stem cells. In this case, the readout is based on the change in the fluorescent signal as a reflection of changes in the expression level of a target protein. Alternative readouts include, without limitation, fluorescence resonance energy transfer, surface plasmon resonance, rolling circle DNA amplification, mass spectrometry, resonance light scattering, and atomic force microscopy. Examples of protein arrays are described in, for example, Zhou H, et al. (2001, Trends Biotechnol. 19: S34-9; Zhu et al., 2001, Current Opin. Chem. Biol. 5: 40-45; Wilson and Nock, 2003, Angew Chem Int Ed Engl 42:494-500; Schweitzer and Kingsmore, 2002, Curr Opin Biotechnol 13:14-9). Biomolecule arrays are also disclosed in U.S. Pat. No. 6,406,921.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

The data disclosed herein demonstrate that cells undergoing differentiation exhibit morphologic changes that are reflected in the cell proteomic profile. The present invention demonstrates that adipogenesis in human adipose-derived adult stem cells is accompanied by modulation of five major protein categories: Cytoskeleton, Metabolic, Heat Shock Protein/Chaperone, Redox, and Protein Degradation (Table 1). The present invention provides a composite profile of the proteomic profile of undifferentiated and differentiated human adipose-derived adult stem cells obtained from multiple donors.

### Example 1

### Liposuction Aspirate Cell Isolation and Culture

The procedures used are modifications of published methods (Aust, et al., 2004, Cytotherapy 6: 1-8; Halvorsen, et al., 2001, Metabolism 50: 407-413; Sen, 2001, J. Cell. Biochem., 81: 312-319; Hauner, et al., 1989, J Clin Invest., 84: 1663-70). Liposuction aspirates from subcutaneous adipose tissue sites were obtained from male and female subjects (n=6) undergoing elective procedures in local plastic surgical offices. The mean age and BMI (±S.D.) of the subjects were 38±14 years and 30.4±7.1, respectively. Tissues were washed 3-4 times with phosphate buffered saline and suspended in an equal volume of PBS supplemented with 1% bovine serum and 0.1% collagenase type I pre-warmed to 37° C. The tissue was placed in a shaking water bath at 37° C. with continuous agitation for 60 minutes and centrifuged for 5 minutes at 300×g at room temperature. The supernatant was removed and the pelleted stromal vascular fraction (SVF) was resuspended in Stromal Medium (DMEM/F12 Ham's, 10% fetal bovine serum, antibiotic/antimycotic and plated at a density of 0.156 milliliter of tissue digest/cm² of surface area in T225 flasks using Stromal Medium for expansion and culture. This initial passage of the primary cell culture is referred to as “Passage 0” (P0). Following the first 48 hours of incubation at 37° C. at 5% CO₂, the cultures were washed with phosphate buffered saline (PBS) and maintained in Stromal Media until they achieved 100% confluence (mean cell density of ˜31,000 cells per cm² after 4.2±1.5 days in culture). The cells were passaged by trypsin/EDTA digestion and seeded at a density of 30,000 cells/cm² (“Passage 1”) on 10 centimeter plates for day 0 protein harvest or on 6 well plates for day 9 adipogenesis protein harvest.

### Cell Culture and Adipogenesis

One day after seeding, plates were either harvested for protein (day 0) or the medium was replaced with an Adipogenic Differentiation Medium composed of DMEM/F-12 with 3% FBS, 33 μM biotin, 17 μM pantothenate, 1 μM bovine insulin, 1 μM dexamethasone, 0.25 mM isobutylmethylxanthine (IBMX), 5 μM rosiglitazone, and 100 U penicillin/100 μg streptomycin/0.25 μg Fungizone. After three days, Adipogenic Differentiation Medium was changed to Adipocyte Maintenance Medium which was identical to the induction media except for the removal of both IBMX and rosiglitazone. Cells were fed every third day and maintained in culture for 9 days prior to protein harvest.

### Oil-Red-O Staining and Quantification

Cells were fixed in 10% formaldehyde/PBS fixative solution for 20 minutes, and then rinsed 5 times with ddH₂0. 0.3% Oil-Red-O (Sigma, St. Louis, Mo.) solution was added to the cells for 2 hours on the orbital shaker. The stain was then aspirated, and the cells were washed with ddH₂0 until no residue remained in the culture plates. The plates were then scanned on a flat bed scanner with photo-quality resolution, and the images were used to quantitate the percentage of cells stained with Oil-Red-O using MetaVue software (Universal Imaging Corp., Downingtown, Pa.).

### Protein Extracts

Cells plated in 10 centimeter or 6 well plates were washed with ice cold PBS and lysed directly in 1 milliliter of Ready Prep Sequential Extraction Reagent 3 prepared according to the manufacturer's (BioRad) instructions. The lysates were sonicated until clear, incubated at room temperature for 1 hour, and centrifuged at 18,000×g for 10 minutes at room temperature. Protein extracts were concentrated using Centricon 10 tubes centrifuged at 5000×g at room temperature. Protein concentrations were determined using the BioRad Protein Assay Reagent and stored at −80° C. prior to use.

### 2 Dimensional-Polyacrylamide Gel Electrophoresis (PAGE)

Protein samples were solubilized in 5 M Urea/2 M ThioUrea/2% CHAPS/2% SB3-10/0.2% Bio-lyte, pH 3-10/5 mM PMSF/2 mM TBP/50 mM DTT/2% n-dodecyl-B-d-maltoside/150 U endonuclease/1× Protease Inhibitor. Following centrifugation to remove unsolubilized material, samples were rehydrated at ˜1 milligram per gel in DeStreak Reagent, (Amersham 17-6003-18, 2-hydroxyethyl disulfide) containing 1% ampholytes, pH 3-10 (BioRad 163-9094) and were introduced into dry IPG strips (typically 24 centimeters, pH 3-10 NL) under conditions of active rehydration (e.g. with a slight voltage applied across the strips). All gels were run in duplicate. Proteins were focused at a maximum 10,000 V for a total of 90,000 v-h. Upon completion of 1st dimension electrophoresis, the IPG strips were either directly subjected to 2nd dimension SDS-PAGE or frozen at −80° C. for later analysis.

For the 2nd dimension, the IPG strips were equilibrated first with 0.375 M Tris-HCl, pH 8.8, 6M urea, 20% glycerol, 2% SDS, 1% DTT for 15 minutes followed by a second equilibration with 0.375M Tris-HCl, pH 8.8, 6M urea, 20% glycerol, 2% SDS, 2.5% iodoacetamide for 15 minutes. The strips were rinsed with electrophoresis buffer (25 mM Tris, 190 mM glycine, 0.1% SDS) and then embedded in low-melting temperature agarose onto the top of 25×20 centimeter 12% acrylamide gel. Gels were run at constant current until the bromophenol blue dye front reached the bottom of the gel and stained with Sypro Ruby. The stained gels were scanned with a Molecular Imager FX with data directly imported into PDQuest. For each gel, the relative abundance of each resolved protein feature was quantified by mathematical fitting of Gaussian curves in two dimensions. Data within each were normalized (expressed as a percentage of total spot abundance) and routine statistical analyses available within the software package were used to identify unique spots, absent spots, or spots up or down regulated under specified conditions.

### Trypsin Digestion

Following electrophoresis, staining, scanning, spot detection, and match set preparation, proteins of interest were selected and their standard spot numbers entered into a “Cut List.” This “Cut List” was used by the automated spot cutter to select and excise the protein features in order of least to must abundant from one or more gels. Excised gel plugs were deposited into a 96 well plate and transferred to the MassPrep (Waters/Micromass) station. Proteins within the gel plugs were automatically destained, reduced, alkylated, dehydrated, rehydrated and digested with trypsin. The resulting peptides were extracted, cleaned-up, and then deposited into 96 well plates for analysis.

### Q-TOF Analysis

The peptides from each digested spot were separated by capillary liquid chromatography interfaced to an ESI-MS/MS MicroMass Q-TOF micro mass spectrometer. MassLynx 4.0 software package (Waters) was used to identify individual mass spectrograms. Parameters included calculation of charge states and peaks were de-isotoped. The ProteinLynxGlobalServer 1.1 software was used to search Release 43.0 of Swiss-Prot containing 146,720 sequence entries for protein identification using 100 ppm precursor-ion and fragment-ion mass accuracy, modifications included phosphorylation, oxidation of methionine, and cysteines modified with iodoacetamide, 1 missed cleavage and using trypsin.

Scores above 100 were generally considered valid identifications, although any identification with a score below 200 was examined carefully, to verify that the spectra included a good number of consecutive “y” ions with high mass accuracy. The number of peptides analyzed and the percentage coverage of the total amino acid sequence was determined for each protein identified. The database was checked for redundancy and inspected for single proteins listed under multiple names. The molecular weight and pI of identified proteins were evaluated and verified relative to the electrophoretic mobility of the protein feature on the 2-dimensional gel. Proteins were classified into functional categories based on their listed description in the Swiss-Prot database.

### Analysis Criteria

The proteomic profile of the undifferentiated and differentiated human adipose-derived adult stem cells was defined based on the following guidelines: (1) Proteins “induced (or upregulated)” or “reduced (or downregulated)” during adipogenesis displayed both a 98% significance in comparisons between replicate groups and >2-fold induction (51 features) or >3-fold reduction (23 features) with differentiation.

### Immunoblotting

The samples were resolved by 12.5% SDS-PAGE and electroblotted onto nitrocellulose. Immunoblot blocking and all subsequent incubations were carried out in a solution of Odyssey blocking buffer (LI-COR Biosciences) diluted 1:1 with phosphate-buffered saline (PBS). The immunoblots were incubated with a panel of anti-heat shock protein antibodies obtained from StressGen Biotechnologies (Victoria, BC): at the indicated dilutions: rabbit polyclonals—anti-calreticulin (SPA-600, 1:2,000), anti-αA/αB-crystallin (SPA-224, 1:1000), anti-phospho crystallin αB (ser19) (SPA-225, 1:1,000), anti-phospho crystalline αB (ser45, 1:1,000) (SPA-226), anti-phospho crystalline αB (ser59) (SPA-227, 1:1,000), anti-GP78 (SPA-826, 1:1000), anti-HSP20 (SPA-796, 1:5,000), anti-HSP27 (SPA-803, 1:5,000), anti-phospho HSP27 (ser 78) (SPA-523, 1:2,000), anti-phospho HSP27 (ser 82) (SPA-524, 1:1,000), antiphospho HSP27 (ser 15) (SPA-525, 1:2,000), anti-HSP60 (SPA-805, 1:1,000), anti-HSP70 (SPA-811, 1:2,000), and anti-HSP90 (SPA-846, 1:1,000); mouse monoclonals—anti-FKBP59 (SPA-1400, 1:5,000) and anti-HSP47 (SPA-470, 1:1,000).

Each rinse step was carried out using phosphate-buffered saline containing 0.1% Tween-20. For detection, the immunoblots were incubated with goat anti-rabbit secondary antibody IR800 (Rockland, Inc. Cat. # 610-132-121) or goat anti-mouse secondary antibody IR800 (Rockland, Inc. Cat # 610-132-122) diluted 1:5,000 in the Odyssey buffer:PBS (1:1) solution and scanned using the Odyssey Infrared Imaging System (LI-COR Biosciences). Signal intensities were quantified using the Un-Scan-It Version 5.1 (Silk Scientific, Inc., Orem, Utah). Relative intensity of the differentiated (D) to undifferentiated (U) samples was determined using the following formula: Relative intensity=(Differentiated pixel count−background)/(Segment area)÷(Undifferentiated pixel count−background)/(Segment area). Values are reported as the average of samples obtained from 2 to 4 donors.

The results of the experiments presented in this Example are now described.

### Adipogenesis

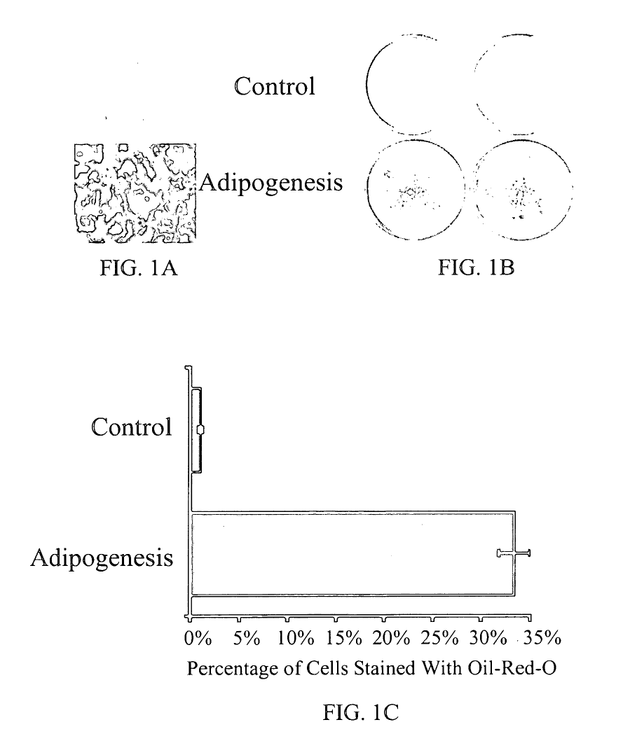

Human adipose-derived adult stem cells isolated from subcutaneous liposuction aspirates of 6 nondiabetic, healthy donors (mean age=38±14.2 years, mean BMI 30.4±7.1) were expanded to “passage 1” and plated at a density of 3×10⁴ cells/cm² in 100 mm plates. Cells from four of the donors (2 female, 2 male) were induced with a combination of adipogenic factors (insulin, isobutylmethylxanthine, dexamethasone, thiazolidinedione) and differentiated for an additional 9 days in culture. Both female and male subjects were included to avoid biasing this analysis of the adipose-derived adult stem cell proteome and to focus on those characteristics shared independent of gender. Adipogenesis was accompanied by the increased appearance of lipid vacuoles staining positive with Oil Red O, indicating the presence of neutral lipids. A representative induction is shown in FIG. 1 at the microscopic and macroscopic scale; over 35% of the surface area stained positive with Oil Red O.

### Undifferentiated Adipose-Derived Adult Stem Proteome

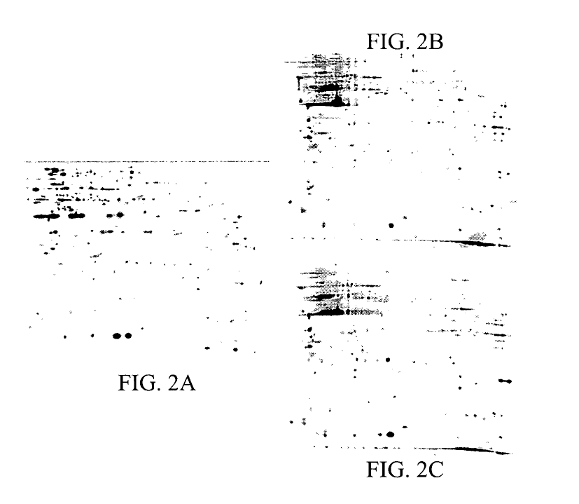

Protein lysates prepared from undifferentiated (n=6 donors) and differentiated (n=4 donors) human adipose-derived adult stem cells were separated by 2-dimensional polyacrylamide electrophoresis and detected with Sypro Ruby (FIG. 2). The number of features identified on each gel ranged from 691 to 795. The undifferentiated gels shared 467 features in common as compared to 434 features on the differentiated gels; a total of 288 features were common to all gels. The data from 4 individual donors was combined to create a “master” map for both the undifferentiated and differentiated human adipose-derived adult stem cells (FIG. 2).

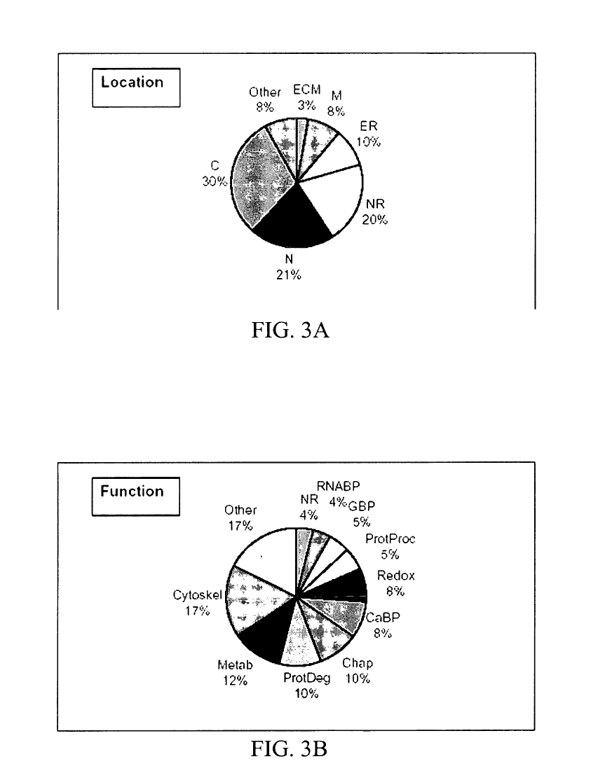

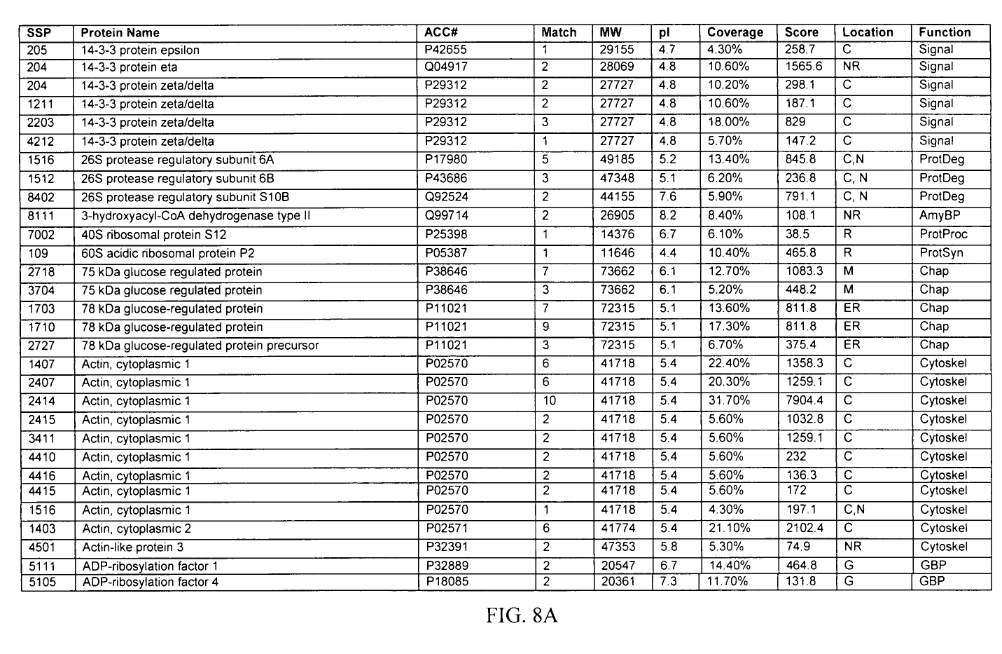

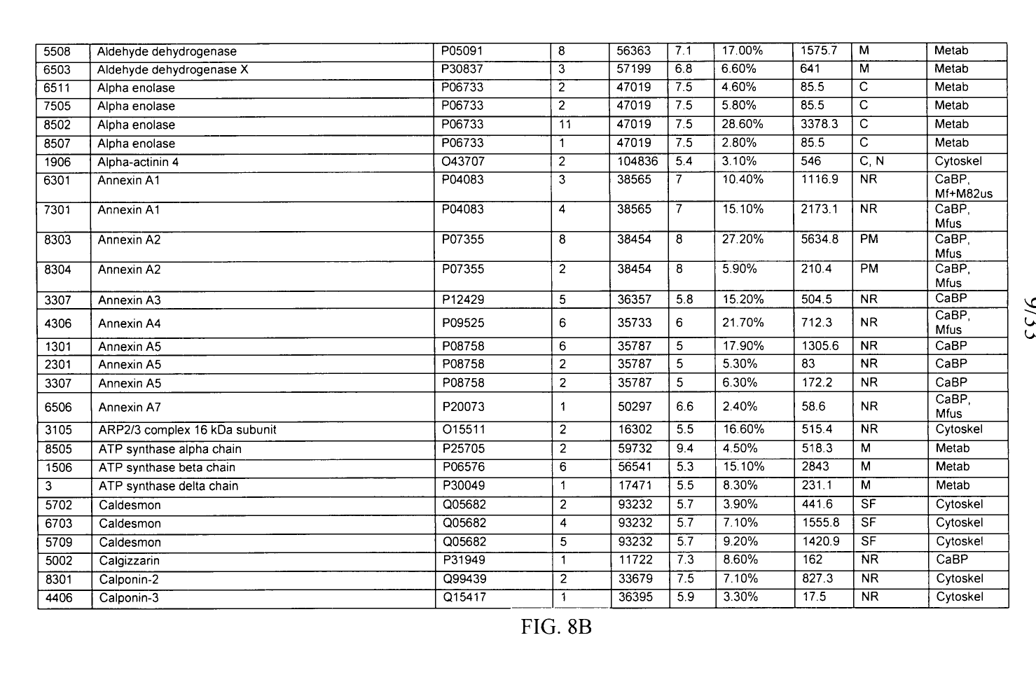

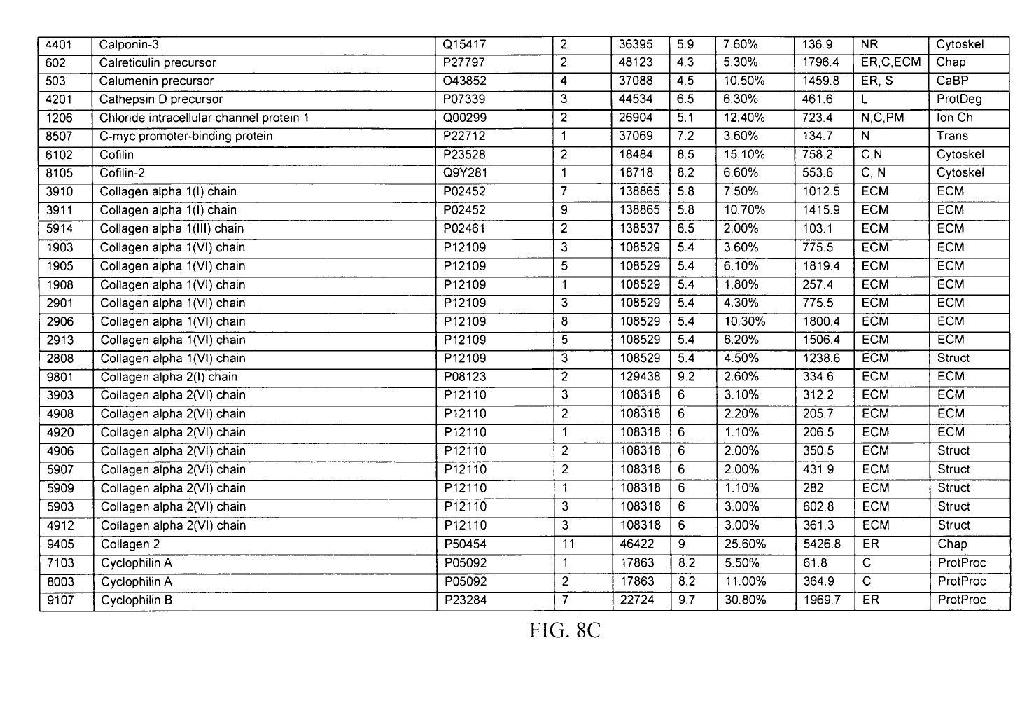

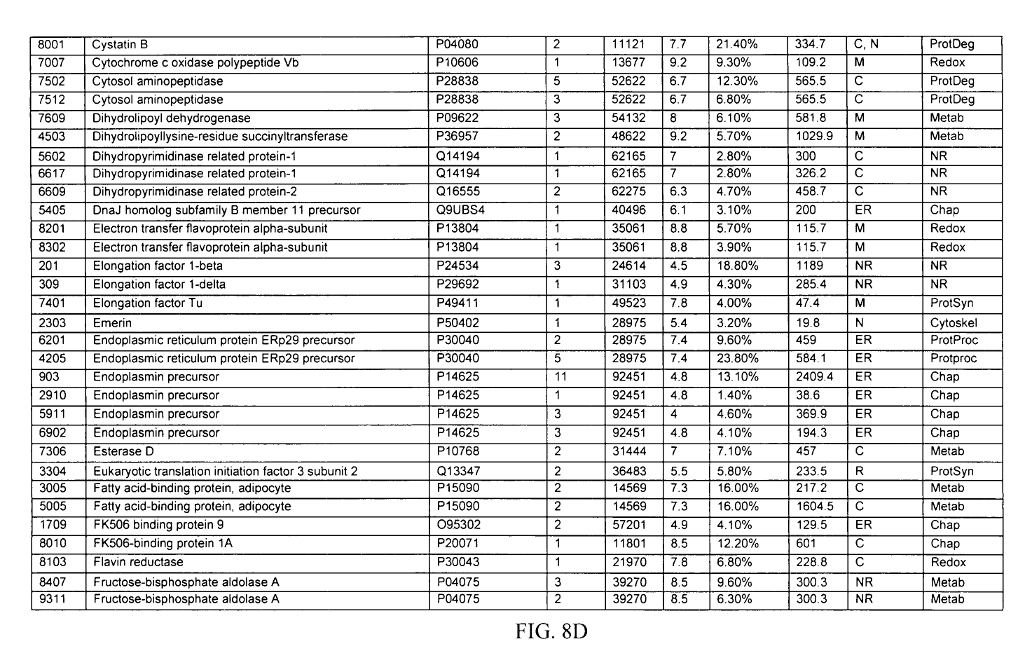

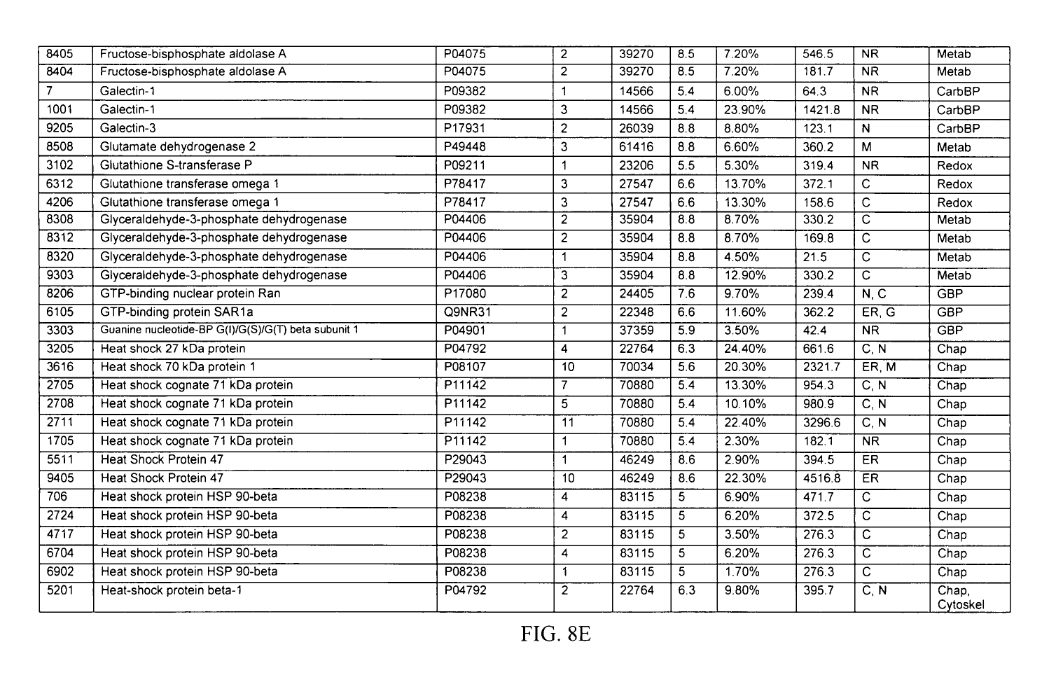

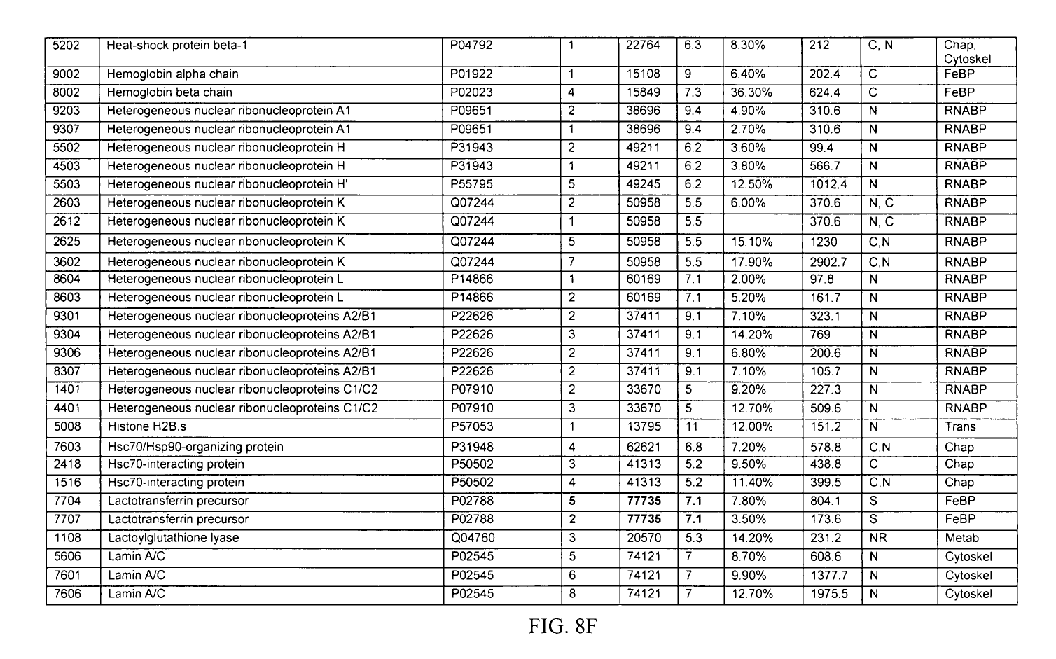

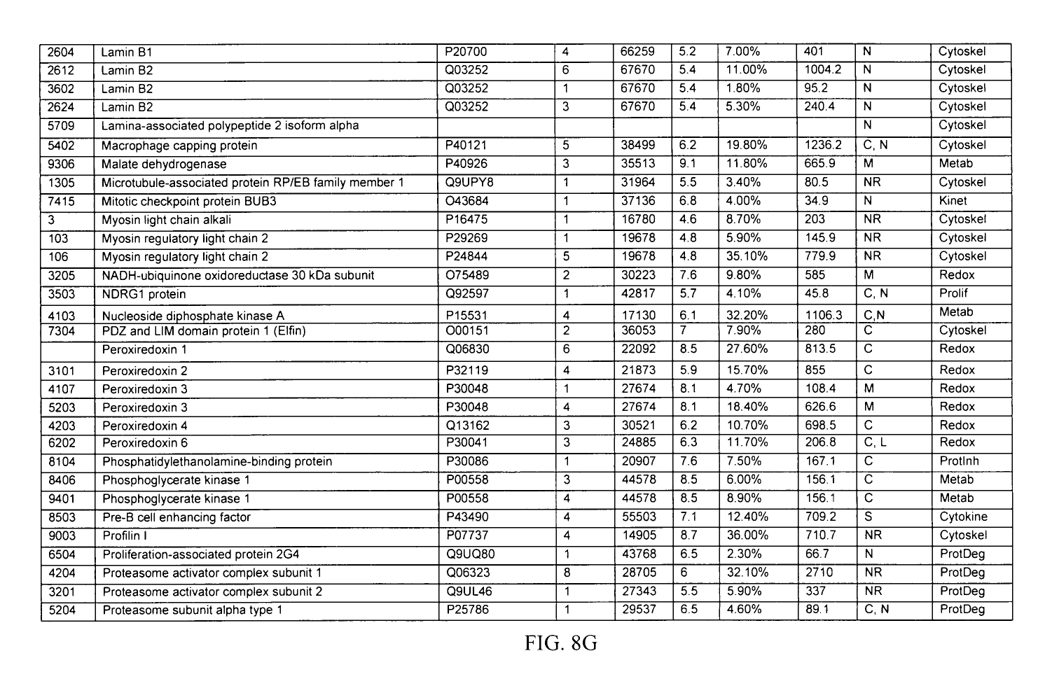

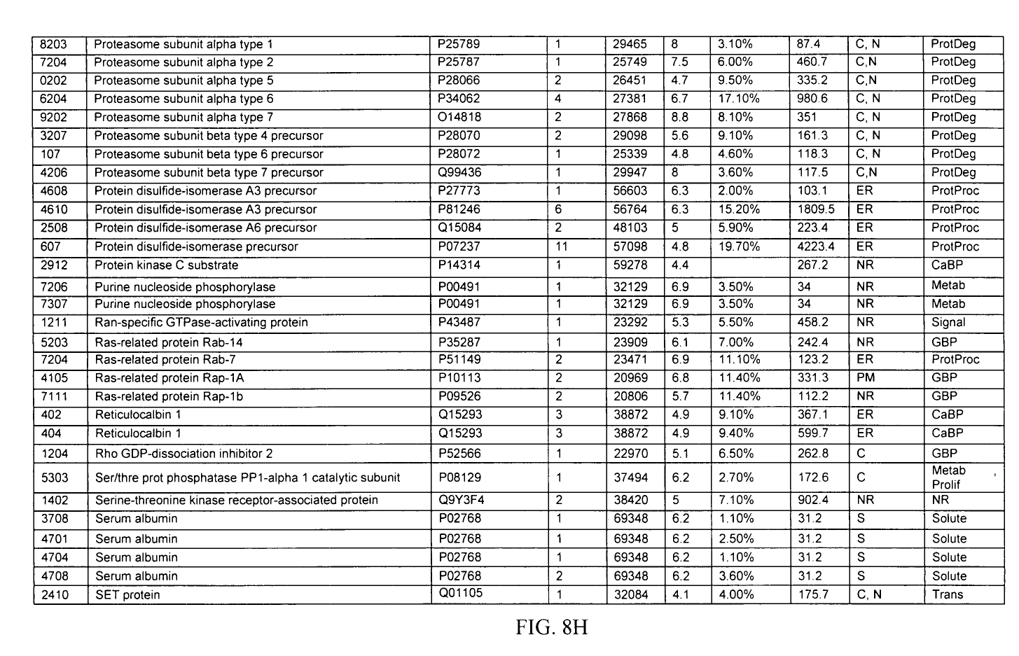

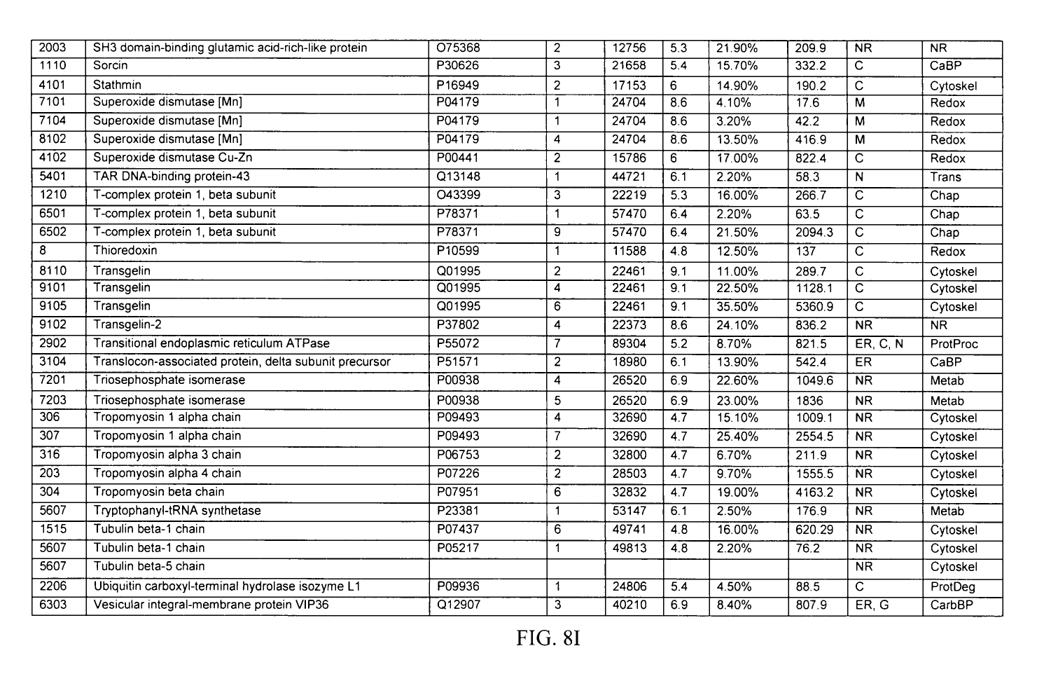

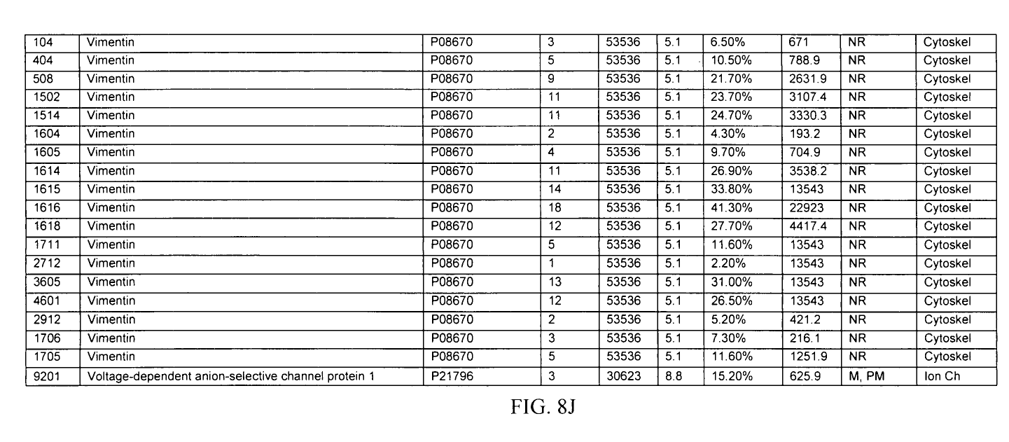

The functionality and subcellular localization of the 175 proteins identified in the undifferentiated adipose-derived adult stem cells are summarized in FIG. 3. The identity, number of peptides matched, percentage coverage, “score”, pI, molecular weight, and accession number of each protein are presented in FIG. 8. The peptide sequences of those features identified by a single peptide match are presented in Table 3.

### Differentiated Adipose-Derived Adult Stem Proteome

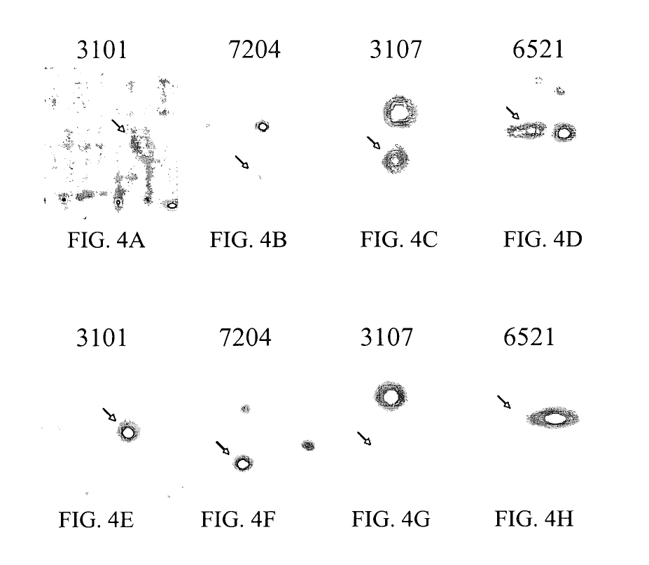

Comparison between the gels identified proteins that were induced by ≧2-fold or reduced by ≧3-fold following differentiation (p<0.02, 98% confidence level). Representative protein features are depicted in FIG. 4. The proteins identified as SSP numbers 3101 and 7204 correspond to fatty acid binding protein-adipocyte and heat shock protein 20-like protein, respectively, and are both induced with adipogenesis. In contrast, the proteins identified as SSP numbers 3107 and 6521, corresponding to Stathmin and elfin (as well as LIM and SH3 domain protein 1) respectively, are reduced with adipogenesis.

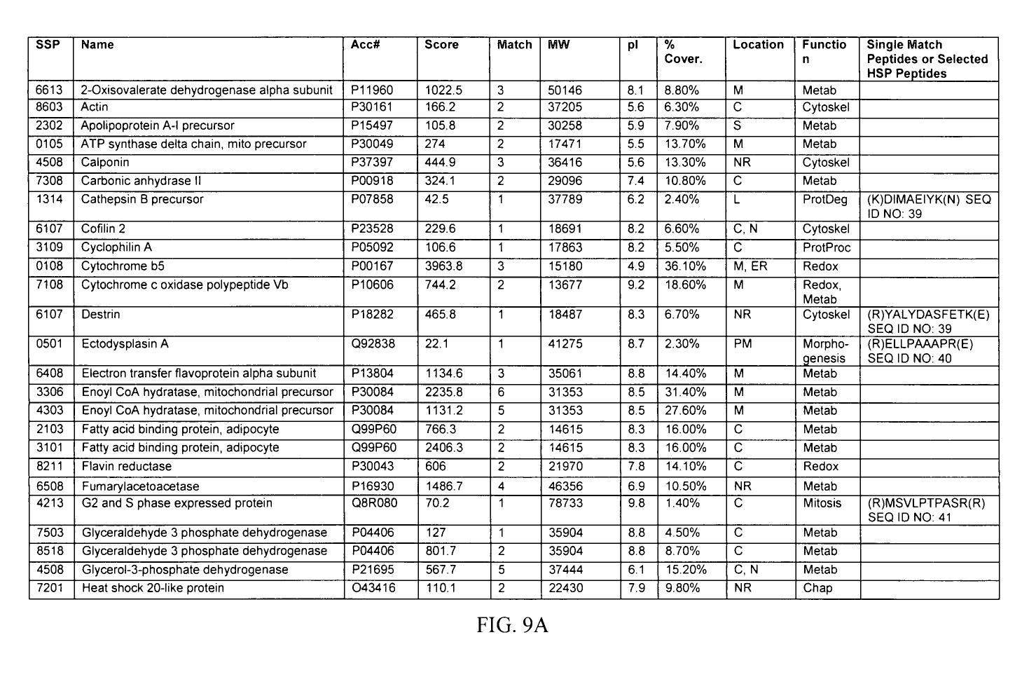

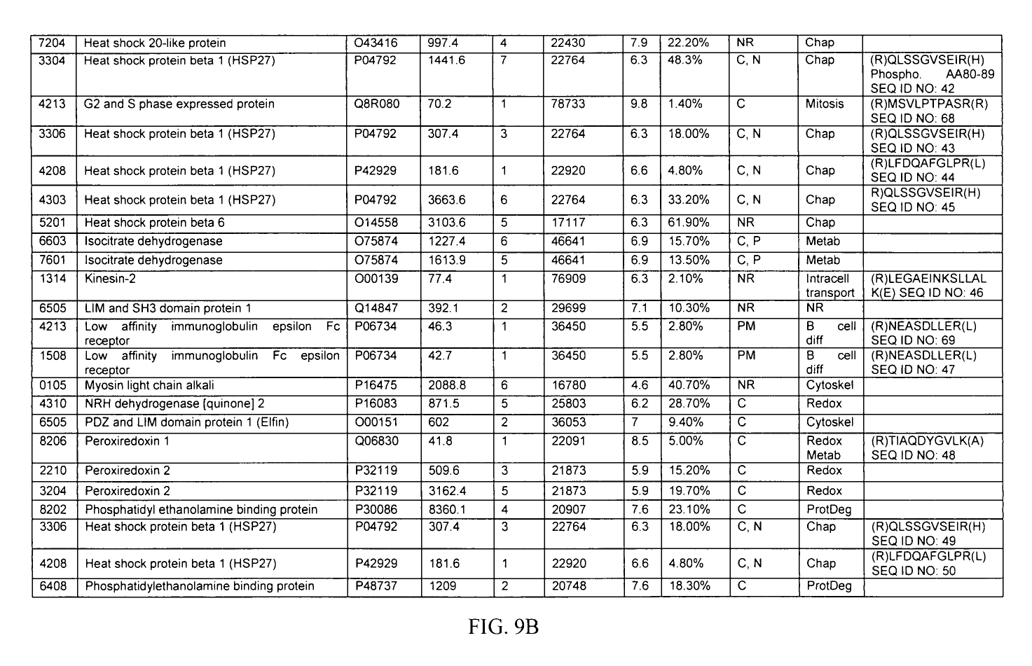

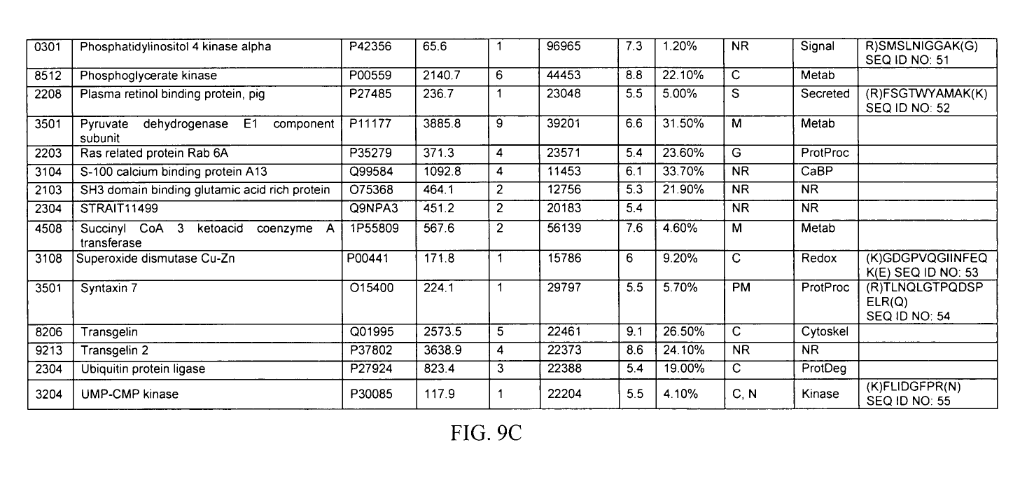

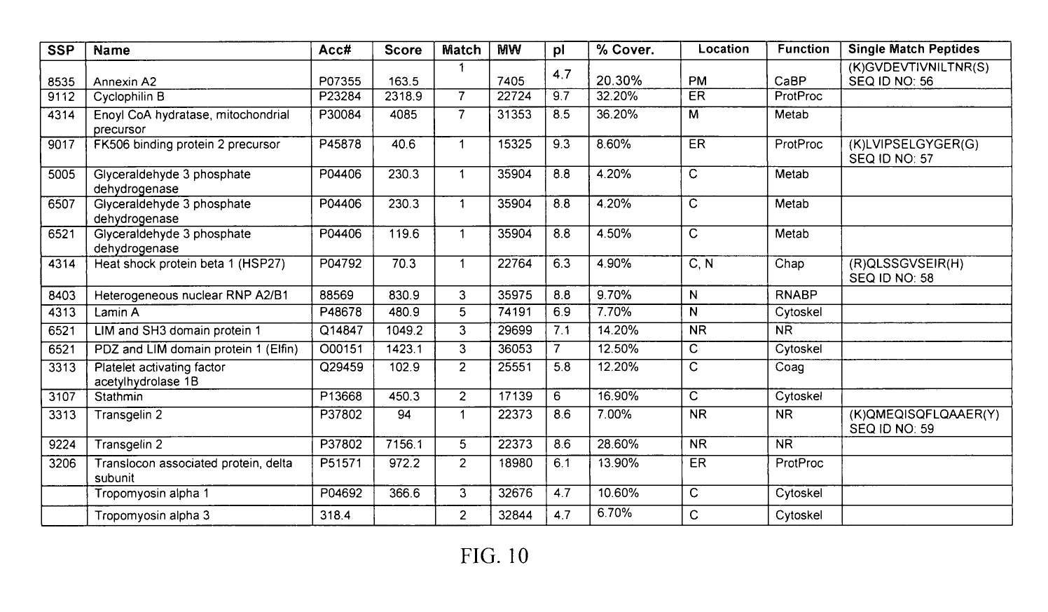

FIG. 9 provides a list of those proteins modulated by adipogenesis, including the number of peptides matched, percent coverage, pI, molecular weight, accession number, score, subcellular location, and function. An abbreviated list of these proteins is presented in Table 1; those proteins uniquely attributed to differentiated human adipose-derived adult stem cells as a result of the present invention are highlighted in bold fonts. Proteins were categorized into functional groups based on their definition within the Swiss-Prot database. Adipogenesis reduced expression of proteins within selected functional classes, including cytoskeletal/structural, metabolic, and heat shock protein/chaperone-related proteins. In addition to these same categories, adipogenesis selectively induced proteins involved in oxidation-reduction and proteasomal degradation and ubiquitination.

The expression alterations in specific families of proteins as a result of adipogenesis is disclosed herein, specifically as the present invention relates to cytoskeleton, metabolic, redox and protein degradation and processing proteins.

The morphology of the adipocyte is significantly different from that of a fibroblast. In fact, mechanical tension, acting through the actin filament complex, can control the differentiation status of adult stromal stem cells (McBeath, et al., 2004, Dev. Cell., 6: 483-495). When spread out on a surface, bone marrow-derived adult stem cells form osteoblasts while, when rounded up, they commit to an adipocyte lineage (McBeath, et al., 2004, Dev. Cell., 6: 483-495). This process can be manipulated through the RhoA protein, a GTPase affecting the actin cytoskeleton (McBeath, et al., 2004, Dev. Cell., 6: 483-495). The current study demonstrates that specific proteins regulating actin polymerization (Cofilin2, destrin) are induced in adipocytes. Previous studies have demonstrated the presence of Cofilin in adipose tissue (Choi, et al., 2003, Biosci. Biotechnol. Biochem., 67: 2262-2265). In addition, there is an induction of cytoskeletal proteins associated with the smooth muscle phenotype (transgelins, myosin light chain alkali). In contrast, individual features identified as elfin, a protein associated with the formation of actin stress fibers in myoblasts, are both induced and reduced with adipogenesis (Kotaka, et al., 2001, J. Cell Biochem., 83: 463-472). There is reduced expression of Stathmin, a tubulin polymerization protein whose absence is associated with arrest of the cell cycle and a failure to undergo mitosis (Rubin, et al., 2004, J. Cell. Biochem. 93: 242-250). The reduction of Stathmin is consistent with the association of adipocyte maturation with cell cycle arrest (Morrison and Farmer, 1999, J. Biol. Chem., 274: 17088-17097).

The primary function of adipocytes is in metabolism. The mature adipocyte stores excess energy in the form of lipid. Consequently, adipogenesis in adipocyte cells is accompanied by the induction of proteins associated with glycolysis and fatty acid metabolism (Glyceraldehyde 3 phosphate dehydrogenase, Isocitrate dehydrogenase, Phosphoglycerate kinase, Pyruvate dehydrogenase). The metabolic proteins carbonic anhydrase II, fatty acid binding protein (adipocyte), and glycerol-3-phosphate dehydrogenase were among the first genes found to be upregulated by adipogenesis in the 3T3-L1 murine pre-adipocyte model (Spiegelman, et al., 1983, J. Biol. Chem., 258: 3-10089; Lynch, et al., 1993, Am. J. Physiol., 265: C234-43). While specific glyceraldehyde-3-phosphate dehydrogenase features were induced with adipogenesis (SSP 7503, 8518), others were reduced (SSP 5005, 6507, 6521, 8535). Enoyl CoA dehydrogenase displayed a similar pattern with evidence of both induced and reduced features following cell differentiation.

Adipogenesis in human adipocyte cells is associated with an induction of multiple proteins associated with oxidation/reduction pathways. While mitochondrial proteins accounted for approximately 8% of the undifferentiated adipocyte cell proteomic profile, they represented >18% of the proteins upregulated with adipogenesis (FIG. 9). Many of these same proteins have been detected in the proteome of mature 3T3-L1 adipocytes (Welsh, et al., 2004, Proteomics 4: 1042-1051; (Wilson-Fritch, et al., 2003, Mol. Cell. Biol., 23: 1085-1094. The mature adipocyte contains an increased number of mitochondria in comparison to fibroblastic cells (Toriyama, et al., 2002, Tissue Eng., 8: 157-165). Recent studies link mitochondrial biogenesis to the etiology of diabetes and it is postulated that the thiazolidinediones, oral anti-diabetic drugs and peroxisome proliferator activated receptor γ ligands, may act in part by regulating mitochondrial formation, especially in brown adipose tissue (Wilson-Fritch, et al., 2003, Mol. Cell. Biol., 23: 1085-1094; Spiegelman, et al., 2000, Int. J. Obes. Relat. Metab. Disord., 24 Suppl 4: S8-10).

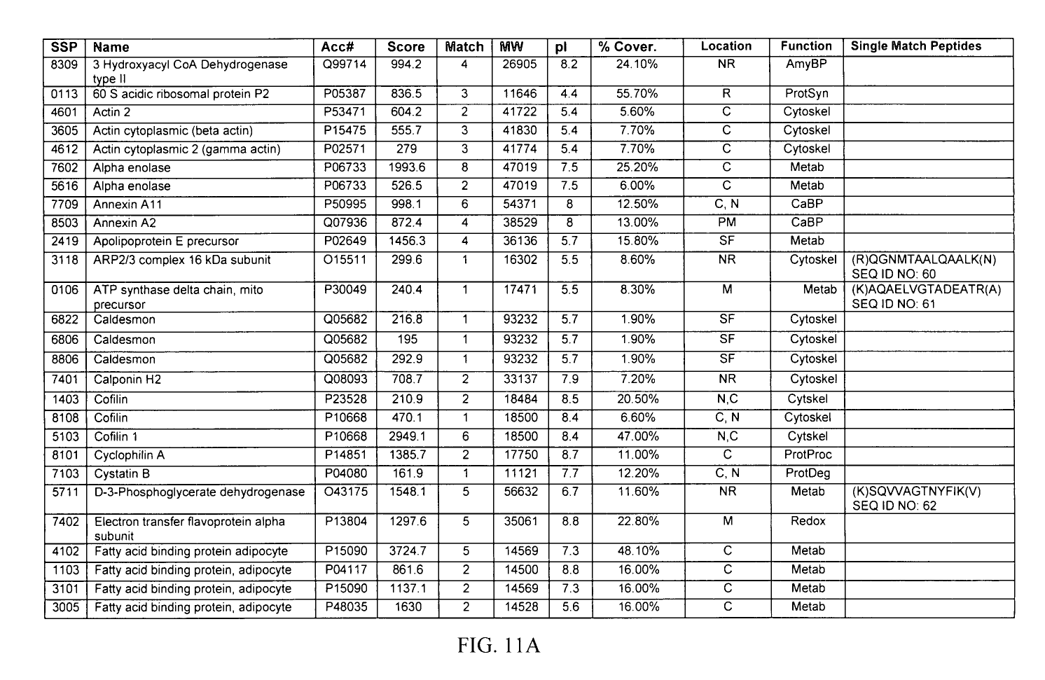

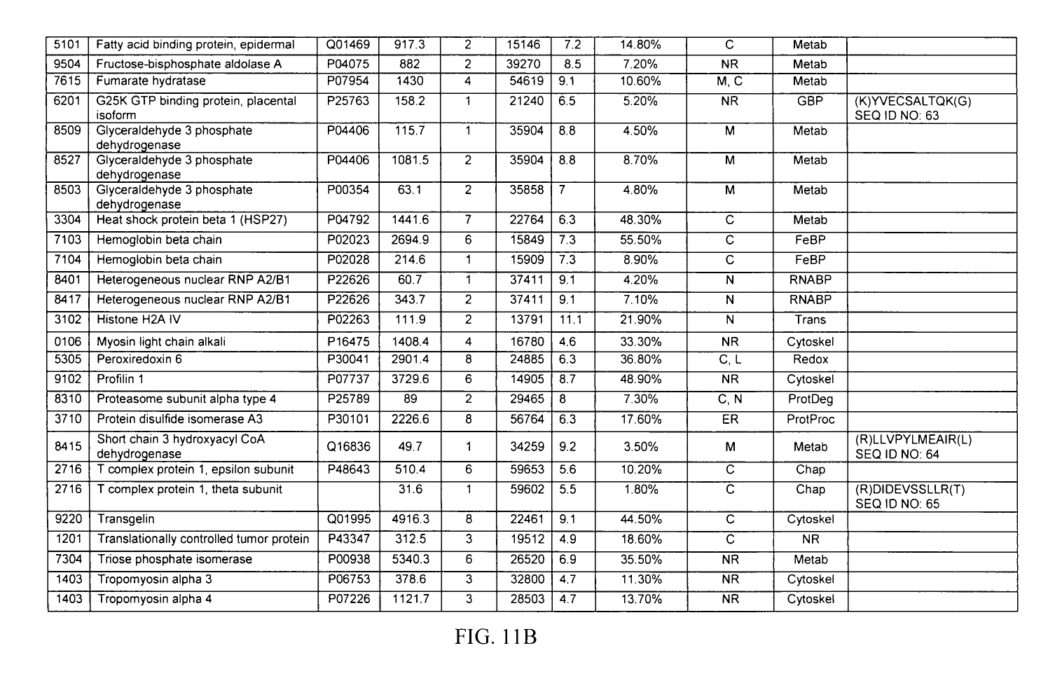

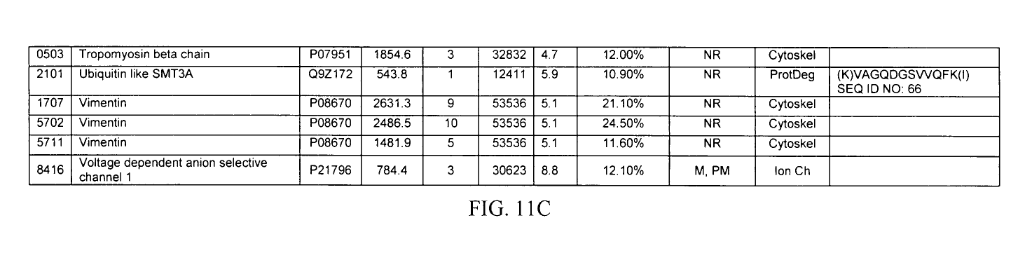

The heat shock proteins/chaperones form complexes that direct translationally-modified proteins to the proteasomal pathway for degradation. For example, the adipogenic transcriptional regulator, the peroxisome proliferator activated receptor γ, is targeted to the proteasome by ubiquinylation and SUMOylation in 3T3-L1 cells (Hauser, et al., 2000, J. Biol. Chem., 275: 18527-18533; Floyd and Stephens, 2002, J. Biol. Chem., 277: 4062-4068; Floyd and Stephens, 2004, Obes. Res. 12: 921-928). Consistent with this is the present observation that human adipocyte cell adipogenesis is associated with induction of an ubiquitin conjugating enzyme (FIG. 8; FIG. 9). In addition, the Ubiquitin-like protein SMT3A or SUMO2 is present in adipocyte cells (FIG. 11). These findings indicates that adipogenesis involves selective modifications of the protein processing and degradation pathways.

### Effect of Differentiation on Heat Shock/Chaperone Proteins

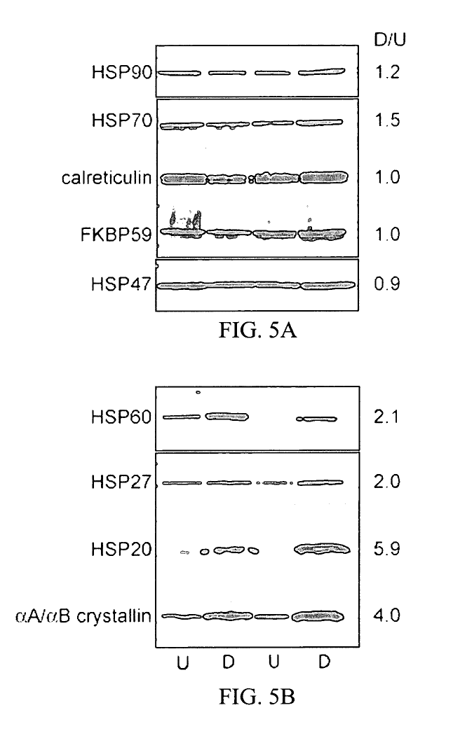

Alterations in the expression of heat shock proteins have been linked to obesity and diabetes. Immunoblots were performed using a panel of antibodies to confirm, validate, and extend the proteomic analysis of the heat shock protein and chaperone family. Control studies documented that each of these antibodies detected an appropriate sized signal in protein lysates prepared from intact human adipose tissue. Two of the protein lysates used in the proteomic analysis described above were examined; both undifferentiated and differentiated human adipose-derived adult stem cells from a male and a female donor were examined. Consistent with the proteomic study, the immunoblots demonstrated an induction of crystallin (heat shock protein β), HSP 20, and HSP 27 with adipogenesis by an average of 4-, 5.9-, and 2-fold, respectively (FIG. 5). In addition, HSP60, which was not detected by the mass spectroscopy analysis, displayed a 2.1-fold induction following adipogenesis. In contrast, the relative levels of the heat shock and chaperone proteins HSP 47, HSP 70, HSP 90, and FK506 Binding Protein showed little or no change following induction of adipogenesis (FIG. 5); each of these proteins had been identified by the proteomic analysis in the undifferentiated adipocyte cells and were not changed following adipogenesis.

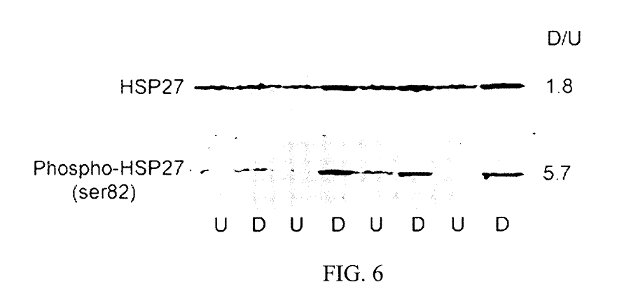

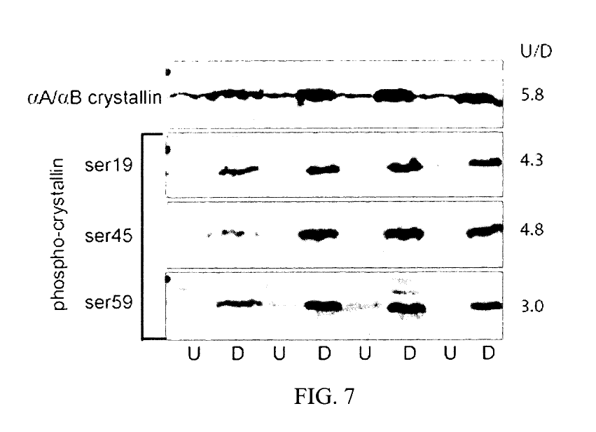

The mass spectrogram of at least one HSP27 peptide (SSP 3304) suggested that the adipogenic-induced protein might be phosphorylated on serine residue 82 (FIG. 9). Immunoblots prepared with the protein extracts prepared from Undifferentiated and Differentiated cells of all four donors used in the proteomic analysis were probed with antibodies detecting all forms of HSP27 and those specific for the HSP27 phosphoserines 82, 15, and 78. No evidence of the HSP 27 phosphoserine 15 or 78 proteins was detected; however, the phosphoserine 82 form of HSP 27 was induced an average of 5.7-fold and this exceeded the 1.8-fold induction of total HSP 27 observed in the same donors (FIG. 6). Similar studies examined the crystallin αB phosphorylation status on identical immunoblots (FIG. 7). The serine residues 19, 45, and 59 of the adipogenic-induced crystallin αB proteins each displayed evidence of phosphorylation; upon differentiation, the levels of these phosphoproteins increased by 4.3-, 4.8-, and 3.0-fold, respectively. All donors displayed similar patterns of induction, although the—fold increase varied between individuals (FIGS. 6 and 7).

The adipogenic induction of crystallin (total and serine phosphoproteins 19, 45, 59), HSP20, HSP27 (total and serine phosphoprotein 82), and HSP60 in human adipocyte cells is intriguing. The heat shock proteins serve as chaperones, controlling protein folding in the endoplasmic reticulum and their subsequent intracellular trafficking (Young, et al., 2004, Nat. Rev. Mol. Cell. Biol., 5: 781-91). There is a growing body of literature linking chaperone-like molecules to adipogenesis, obesity, and diabetes (Cherian, et al., 1995, Biochem. Biophys. Res. Commun., 212: 184-189; Kumar, et al., 2004, Biochem. J., 379: 273-282; Kurucz, 2002, Diabetes 51: 1102-1109; Ozcan, et al., 2004, Science 306: 457-461). For example, adipogenesis in 3T3-L1 cells is accompanied by increased expression of the chaperone-related immunophilin, FK Binding protein 51 (Yeh, et al., 1995, Proc. Natl. Acad. Sci. U.S.A., 92: 11081-11085). Moreover, the nuclear hormone receptors that control adipogenic transcription, the glucocorticoid receptor and the peroxisome proliferator activated receptor, are sequestered in the cytosol as a complex with heat shock proteins HSP 70 and HSP90 prior to ligand activation (Young, et al., 2004, Nat. Rev. Mol. Cell. Biol., 5: 781-91; Hache, et al., 1999, J. Biol. Chem., 274: 1432-1439; Sumanasekera, et al., 2003, J. Biol. Chem. 278: 4467-73. Clinical studies have linked polymorphisms in HSP70 to an increased risk for obesity and type-2 diabetes (Chouchane, et al., 2001, Int. J. Obes. Relat. Metab. Disord., 25: 462-466; Zouari Bouassida, et al., 2004, Diabetes Metab., 30: 175-180. It is postulated that obesity leads to insulin resistance and diabetes by causing endoplasmic reticulum stress (Ozcan, et al., 2004, Science, 306: 457-461). This stress has been found to interfere with the serine/threonine phosphorylation-mediated signal transduction pathway downstream of the insulin receptor (Ozcan, et al., 2004, Science, 306: 457-461). Consistent with this is the independent observation that the heat shock protein HSP27 interacts with the insulin-like growth factor receptor 1 and its signal transducer, the serine/threonine kinase protein akt, which together modulate adipocyte metabolism (Rane, et al., 2003, J. Biol. Chem., 278: 27828-27835; Shan, et al., 2003, J. Biol. Chem., 278: 45492-45498). Diabetes alters the metabolism of the chaperone crystallin α, increasing its glycation status (Cherian, et al., 1995, Biochem. Biophys. Res. Commun., 212: 184-189; Kumar, et al., 2004, Biochem. J., 379: 273-282). In the lens of the eye, this biochemical change contributes to cataract formation.

Phosphorylation of crystallin α alters its subcellular localization and its ability to associate with an adaptor protein of the ubiquitin protein isopeptide ligase (den Engelsman, et al., 2004, Eur. J. Biochem., 271: 4195-4203). In cardiomyocytes, crystallin α phosphorylation correlates with inhibition of caspase activity and protects the cell from apoptotic events (Morrison, et al., 2003, Circ. Res., 92: 203-211). The present disclosure demonstrates that adipogenesis enhances expression of these protective forms of crystallin α in human adipocyte cells.

TABLE 1
Functional Categories of Human adipocyte
Cell Proteins Modulated During Adipogenesis

| Protein Function | | |
|---|---|---|
| Categories | Induced > 2-fold (n = 45) | Reduced > 3-fold (n = 13) |
| Metabolism | Apolipoprotein A-1, ATP synthase Δ | Glyceraldehyde 3- |
| | chain, Carbonic anhydrase II, Electron | Phosphate, Dehydrogenase, |
| | transfer flavoprotein alpha-subunit, | Enoyl CoA hydratase |
| | Enoyl CoA Hydratase, Fatty Acid | |
| | Binding Protein (Adipocyte), | |
| | Fumarylacetoacetase, Glyceraledehyde- | |
| | 3-phosphate dehydrogenase, Glycerol- | |
| | 3-phosphate dehydrogenase [NAD+], | |
| | cytoplasmic, Isocitrate Dehydrogenase, | |
| | Phosphoglycerate kinase 1, Pyruvate | |
| | Dehydrogenase D-3, 2-Oxisovalerate | |
| | Dehydrogenase α subunit, Succinyl | |
| | CoA ketoacid coenzyme A transferase 1 | |
| Heat Shock/ | Cyclophilin A, HSPβ6 (crystallin), HSP | Cyclophilin B, FK Binding |
| Chaperones | 20-like protein, HSP27 | protein 2, HSP27 |
| Redox | Cytochrome b5, Cytochrome c oxidase | |
| | polypeptide Vb, Flavin Reductase, NRH | |
| | Dehydrogenase, Peroxiredoxin 1 | |
| | (Thioredoxin peroxidase 2), | |
| | Peroxiredoxin 2, Superoxide Dismutase | |
| | Cu—Zn | |
| Cytoskeleton | Actin, Cofilin 2, Destrin (Actin- | Elfin, Lamin A, Stathmin, |
| | depolymerizing factor), Elfin, Myosin | Transgelin 2, Tropomyosin 1 |
| | light chain alkali, Transgelin | α Chain, Tropomyosin 3 α |
| | | Chain |
| Protein | Cathepsin B, Ubiquitin protein ligase, | |
| Degradation | Phosphatidylethanolamine-binding | |
| and Processing | protein, Ras-related protein Rab-6A, | |
| | Syntaxin 7 | |
| Serine Protease | PAI-1, PEDF, placental thrombin inhibitor, | |
| Inhibitors | pregnancy zone protein, protease C1 | |
| (Serpins) | inhibitor (C1 inh) | |
| Other | Ectodysplasin A, G2 and S phase | Annexin 2, hnRNP A2/B1, |
| | expressed protein, Kinesin 2, LIM and | Translocon associated |
| | SH3 domain protein 1, Low affinity Ig | protein δ, Platelet activating |
| | Fc ε receptor, phosphatidyl inositol 4 | factor acetylhydrolase 1B |
| | kinase α, plasma retinal binding protein, | |
| | S100 calcium-binding protein A13, SH3 | |
| | domain binding glutamic rich protein, | |
| | STRAIT11499, Transgelin 2, UMP- | |
| | CMP kinase | |

TABLE 2
Summary of Published Proteomic Analyses of
Fibroblasts, Adipocytes, and Related Cells and Tissues

| | | No. of Distinct Proteins | | |
|---|---|---|---|---|
| | Induction | Identified by MS (% | No. of MS | No. of MS |
| Cell or | process or | similarity or identity to human | Identified | Identified |
| Tissue Type | subcellular | adipocyte | Protein Spots | Protein Spots |
| (Species) | fractionation | proteome)* | Induced | Reduced |
| 3T3-L1 | MDI | 8 (87%) | 7 | 1 |
| (murine) | MDI, | 23 (57%) | | |
| | mitochondria | | | |
| | | 100 (42%) | 40 | 60 |
| | Secreted | 20 (20%) | 8 | |
| White | ob/ob mice | 7 (86%) | 3 | 4 |
| Adipose | high fat diet | 2 (50%) | 0 | 2 |
| (murine) | insulin | 27 (52%) | 3 | 6 |
| | high fat diet | 10 (20%) | 6 | 4 |
| Adipocytes | (caveolae) | 26 (23%) | | |
| (human) | | | | |
| Bone | (TGFβ) | 27 (56%) | 9 | 13 |
| Marrow | | | | |
| MSCs | | | | |
| (human) | | | | |
| Dermal | (aging) | 24 (58%) | | |
| Fibroblasts | | | | |
| (human) | | | | |
| Synovial | | 155 (59%) | | |
| Fibroblasts | | | | |
| (human) | | | | |
| Monocyte/ | (LPS) | 17 (47%) | 10 | 3 |
| Macrophages | (PMA) | 22 (64%) | 20 | 5 |
| (human) | | | | |

*This percentage value reflects those proteins that are identical or similar (belong to a related or the same protein family) to those identified in the present analysis of the human adipocytecell proteome.
Abbreviations:
MDI, Methylisobutylxanthine/dexamethasone/insulin;
LPS, Lipopolysaccharide;
PMA, Phorbol Myristic Acid;
TGFβ, Transforming Growth Factor β

TABLE 3

Sequences for Undifferentiated Human Adipocyte
Cell Single Peptide Matches

| | | | |
|---|---|---|---|
| SSP | Protein Name | ACC# | Peptide |

| | | | |
|---|---|---|---|
| 205 | 14-3-3 protein | P42655 | (K)EAAENSLVAYK(A) |
| | epsilon | | (SEQ ID NO: 1) |
| 7002 | 40S ribosomal | P25398 | (K)DVIEEYF(C) |
| | protein S12 | | (SEQ ID NO: 2) |
| 109 | 60S acidic | P05387 | (K)NIEDVIAQGIGK(L) |
| | ribosomal | | (SEQ ID NO: 3) |
| | protein P2 | | |
| 6506 | Annexin A7 | P20073 | (R)EFSGYVESGLK(T) |
| | | | (SEQ ID NO: 4) |
| 3 | ATP synthase | P30049 | (K)AQAELVGTADEATR(A) |
| | delta chain | | (SEQ ID NO: 5) |
| 5002 | Calgizzarin | P31949 | (K)DGYNYTLSK(T) |
| | | | (SEQ ID NO: 6) |
| 8507 | C-myc promo- | P22712 | (R)YISPDQLADLYK(S) |
| | ter-binding | | (SEQ ID NO: 7) |
| | protein | | |
| 8105 | Cofilin-2 | Q9Y281 | R)YALYDATYETK(E) |
| | | | (SEQ ID NO: 8) |
| 5405 | DnaJ homolog | Q9UBS4 | (K)QYDTYGEEGLK(D) |
| | subfamily B | | (SEQ ID NO: 9) |
| | member 11 | | |
| | precursor | | |
| 309 | Elongation | P29692 | (R)IASLEVENQSLR(G) |
| | factor 1-delta | | (SEQ ID NO: 10) |
| 7401 | Elongation | P49411 | (R)TIGTGLVTNTLAMTEEEK(N) |
| | factor Tu | | (SEQ ID NO: 11) |
| 2303 | Emerin | P50402 | (K)IFEYETQR(R) |
| | | | (SEQ ID NO: 12) |
| 8010 | FK506-binding | P20071 | (-)GVQVETISPGDGR(T) |
| | protein 1A | | (SEQ ID NO: 13) |
| 8103 | Flavin | P30043 | (R)NDLSPTTVMSEGAR(N) |
| | reductase | | (SEQ ID NO: 14) |
| 3102 | Glutathione S- | P09211 | (-)PPYTVVYFPVR(G) |
| | transferase P | | (SEQ ID NO: 15) |
| 3303 | Guanine nucle- | P04901 | (R)LFVSGACDASAK(L) |
| | otide-binding | | (SEQ ID NO: 16) |
| | protein G(I)/ | | |
| | G(S)/G(T) beta | | |
| | subunit 1 | | |
| 9002 | Hemoglobin | P01922 | (R)MFLSFPTTK(T) |
| | alpha chain | | (SEQ ID NO: 17) |
| 5008 | Histone H2B.s | P57053 | (K)AMGIMNSFVNDIFER(I) |
| | | | (SEQ ID NO: 18) |
| 1305 | Microtubule- | Q9UPY8 | (K)FFDANYDGK(D) |
| | associated | | (SEQ ID NO: 19) |
| | protein RP/EB | | |
| | family member | | |
| | 1 | | |
| 7415 | Mitotic check- | O43684 | (K)LNQPPEDGISSVK(F) |
| | point protein | | (SEQ ID NO: 20) |
| | BUB3 | | |
| 3 | Myosin light | P16475 | (R)ALGQNPTNAEVLK(V) |
| | chain alkali | | (SEQ ID NO: 21) |
| 3503 | NDRG1 protein | Q92597 | (R)EMQDVDLAEVKPLVEK(G) |
| | | | (SEQ ID NO: 22) |
| 8104 | Phosphatidyl- | P30086 | (K)LYTLVLTDPDAPSR(K) |
| | ethanolamine- | | (SEQ ID NO: 1) |
| | binding | | |
| | protein | | |
| 6504 | Proliferation- | Q9UQ80 | (K)EGEFVAQFK(F) |
| | associated | | (SEQ ID NO: 23) |
| | protein 2G4 | | |
| 107 | Proteasome | P28072 | (R)LAAIAESGVER(Q) |
| | subunit beta | | (SEQ ID NO: 24) |
| | type 6 | | |
| | precursor | | |
| 7206 | Purine | P00491 | (K)VIMDYESLEK(A) |
| | nucleoside | | (SEQ ID NO: 25) |
| | phosphorylase | | |
| 5303 | Serine/threo- | P08129 | (K)LNLDSIIGR(L) |
| | nine protein | | (SEQ ID NO: 26) |
| | phosphatase | | |
| | PP1-alpha 1 | | |
| | catalytic | | |
| | subunit | | |
| 5401 | TAR DNA-bind- | Q13148 | (R)FTEYETQVK(V) |
| | ing protein-43 | | (SEQ ID NO: 27) |
| 5607 | Tryptophanyl- | P23381 | (R)DMNQVLDAYENK(K) |
| | tRNA | | (SEQ ID NO: 28) |
| | synthetase | | |
| 8 | Thioredoxin | P10599 | (K)TAFQEALDAAGDK(L) |
| | | | (SEQ ID NO: 29) |
| 2206 | Ubiquitin car- | | (K)LGFEDGSVLK(Q) |
| | boxyl-terminal | | (SEQ ID NO: 30) |
| | hydrolase | | |
| | isozyme L1 | | |

### Example 2

Adipocytes secrete multiple growth factors, termed “adipokines”, that exert both local (paracrine) and systemic (endocrine) effects on metabolism. In addition to leptin, these include adiponectin, plasminogen activator inhibitor-1 (PAI-1), resistin, visfatin (pre-B cell enhancing factor), and vaspin (visceral adipose tissue-derived serine protease inhibitor).

The murine pre-adipocyte 3T3-L1 cell line has been the model system for the majority of global analyses of the adipocyte secretome. Nevertheless, there is evidence that adipogenesis may differ between human and murine systems. A case in point is the resistin gene, which was first identified in the 3T3-L1 cells. While in vivo analyses in mice have demonstrated a correlation between serum resistin levels, obesity, and type 2-diabetes, human clinical studies do not show a comparable association in non-obese and obese subjects. Consequently, there is a need to directly examine the secretome in human preadipocyte cell models.

Adipose derived adult stem cells, provide an in vitro method for such studies. The adipose-derived adult stem cells are isolated from subcutaneous human liposuction aspirates by sequential collagenase digestion, differential centrifugation, plastic adherence, and expansion in culture. A single ml of tissue yields ˜250,000 cells within a 6-7 day culture period with a distinct immunophenotype. The human adipose-derived adult stem cells are multipotent, differentiating along the adipocyte, chondrocyte, myogenic, neuronal, and osteoblast lineages. Following induction with dexamethasone, insulin, isobutylmethylxanthine and rosiglitazone, the ASCs accumulate Oil Red O positive lipid vacuoles and display characteristics of mature adipocytes. As disclosed elsewhere herein, human adipose-derived adult stem cells were used to examine changes in the cellular proteome as a consequence of cell differentiation. As demonstrated by the data disclosed herein, adipose-derived adult stem cell differentiation correlated with an induction of chaperones and heat shock proteins, as well as visfatin and pre-B cell enhancing factor. As disclosed in the present Example, 2-dimensional electrophoresis/tandem mass spectroscopy was used to compare the secretome of undifferentiated and differentiated human adipose-derived adult stem cells. In parallel, the corresponding transcriptome was analyzed.

Studies of adipogenic protein induction have demonstrated adipose tissue's role as an endocrine organ. Adipocyte-derived “adipokines” such as adiponectin, leptin, and vaspin (visceral adipose tissue-derived serine protease inhibitor) exert hormone-like activities at the systemic level. The data disclosed herein is based on an examination of the secretome (secreted proteins) of primary cultures of human subcutaneous adipose-derived adult stem cells. Conditioned media obtained from four individual female donors after culture in uninduced or adipogenic induced conditions was compared by 2-dimensional gel electrophoresis and tandem mass spectroscopy. Over 80 individual protein features demonstrating ≧2-fold relative differences were examined. Approximately 47% of the 94 identified proteins were shared with the proteomes of interstitial fluid derived from human mammary gland adipose tissue and the total protein lysates of human adipose-derived adult stem cells. Likewise, 17% of the identified proteins were present in the secretome of murine 3T3-L1 adipocytes and 15% were present in compiled human serum. The secretome included proteins, such as actin and lactate dehydrogenase, that lack a leader sequence or transmembrane domain and are classified as “cytoplasmic” in origin. Nevertheless, a number of established adipokines were detected, such as adiponectin and plasminogen activator inhibitor 1 (PAI-1). Of particular interest was the presence of multiple serine protease inhibitor proteins (serpins). In addition to PAI-1, these included pigmented epidermal derived factor (PEDF), placental thrombin inhibitor, pregnancy zone protein, and protease C1 inhibitor (C1 inh).

### Liposuction Aspirate Cell Isolation and Culture

The procedures used are modifications of published methods {Aust, et al., 2004, Cytotherapy 6: 1-8; Halvorsen, et al., 2001, Metabolism 50: 407-413; Hauner, et al., 1989, J. Clin. Invest., 84: 1663-1670; Delany, et al., 2005, Mol. Cell. Proteomics, 4: 731-740. Liposuction aspirates from subcutaneous adipose tissue sites were obtained from female subjects (n=4) undergoing elective procedures in local plastic surgical offices. The mean age and BMI (±S.D.) of the subjects were 37.0±3.9 years and 25.7±3.8, respectively. Tissues were washed 3-4 times with phosphate buffered saline and suspended in an equal volume of PBS supplemented with 1% bovine serum and 0.1% collagenase type I pre-warmed to 37° C. The tissue was placed in a shaking water bath at 37° C. with continuous agitation for 60 minutes and centrifuged for 5 minutes at 300×g at room temperature. The supernatant was removed and the pelleted stromal vascular fraction (SVF) was resuspended in Stromal Medium (DMEM/F12 Ham's, 10% fetal bovine serum, antibiotic/antimycotic and plated at a density of 0.156 ml of tissue digest/square cm of surface area in T225 flasks using Stromal Medium for expansion and culture. This initial passage of the primary cell culture is referred to as “Passage 0” (P0).

Following the first 48 hours of incubation at 37° C. at 5% CO₂, the cultures were washed with PBS and maintained in Stromal Media until they achieved 80-90% confluence (30,166±3816 cells/cm²). The cells from each donor were passaged by trypsin digestion and seeded at a density of 30,000 cells/cm² (“Passage 1”) on six 48 well plates.

### Adipogenic Cell Culture

Four days after seeding, three plates (uninduced) were maintained in Stromal Medium and fed with this medium every third day. The remaining three plates (induced) were fed with an Adipogenic Differentiation Medium composed of DMEM/F-12 with 3% FBS, 33 μM biotin, 17 μM pantothenate, 1 μM bovine insulin, 1 μM dexamethasone, 0.25 mM isobutylmethylxanthine (IBMX), 5 μM rosiglitazone, and 100 U penicillin/100 μg streptomycin/0.25 μg Fungizone. After three days, Adipogenic Differentiation Medium was changed to Adipocyte Maintenance Medium, which was identical to the induction media except for the removal of both IBMX and rosiglitazone, and fed every third day. On day 9 following induction, the media was removed from both the uninduced and induced plates and replaced with Serum Free Medium (DMEM/F12, 1% antibiotic/antimycotic). After one hour, the medium was removed and discarded. Fresh Serum Free Medium was added to each well and the plates were incubated overnight (16 hours), after which time all uninduced and induced cell conditioned medium for each donor lot was collected, pooled, adjusted to a final concentration of 2 mM PMSF by addition of a 100× stock solution, snap frozen in liquid nitrogen, and stored at −80° C. for future analysis.

Two plates of cells from the uninduced and induced were harvested for total RNA using TRI REAGENT® (100 μl per well) according to the manufacturer's instructions (Molecular Research Center, Cincinnati Ohio). One plate of cells under each condition was harvested for total cell protein by addition of 100 μl per well of IP buffer (1% Triton X-100, 150 mM NaCl, 10 mM Tris-Cl pH 7.4, 1 mM EDTA, 1 mM EGTA, 0.5% Ipegal, protease inhibitors, 2 mM PMSF). Protein and RNA samples were stored at −80° C. for future analysis.

### Conditioned Medium Concentration

Frozen volumes of conditioned medium (35-40 ml of each) were removed from −80° C. and thawed at 4° C. As the samples thawed, a fresh aliquot of 100×PMSF (200 mM) was added to each. Fifteen ml aliquots of the solutions were placed in an Amicon Ultra-15 (catalogue # UFC900524) with a 5,000 molecular weight cut off and centrifuged for 30 minutes at 4,000 rpm and the volumes concentrated between 40- to 200-fold. Protein concentrations of the resulting concentrates were determined (BioRad Protein Assay) and ranged from 0.19 to 4.78 μg/μl.

### 2 Dimensional-Polyacrylamide Gel Electrophoresis (PAGE)

Protein samples were solubilized in 8M urea, 4% CHAPS, 65 mM DTT, 40 mM Tris. Following centrifugation to remove unsolubilized material, 45-70 μg of protein were mixed with rehydration buffer (8M urea, 4% CHAPS, 1% IPG buffer, 0.3% DTT) and were introduced into the dry IPG strips (typically 18 cm, pH 4-10NL) under conditions of active rehydration (e.g. with a slight voltage applied across the strips). All gels were run in duplicate. Proteins were focused at a maximum 10,000 V for a total of 90,000 v-h. Upon completion of 1st dimension electrophoresis, the IPG strips were either directly subjected to 2nd dimension SDS-PAGE or frozen at −80° C. for later analysis. For the 2nd dimension, the IPG strips were equilibrated first with 50 mM Tris-HCl, pH 8.8, 6M urea. 30% glycerol, 2% SDS, 1% DTT for 15 minutes followed by a second equilibration with 50 mM Tris-HCl, pH 8.8, 6M urea, 30% glycerol, 2% SDS, and 5% iodoacetamide for 15 minutes. The strips were rinsed with electrophoresis buffer (25 mM Tris, 190 mM glycine, 0.1% SDS) and then embedded in low-melting temperature agarose onto the top of 25×20 cm 12% acrylamide gel. Gels were run at constant voltage until the bromophenol blue dye front reached the bottom of the gel and stained with Sypro Ruby. The stained gels were scanned with a Molecular Imager FX (BioRad, Hercules, Calif.) with data directly imported into PDQuest. For each gel, the relative abundance of each resolved protein feature was quantified by mathematical fitting of Gaussian curves in two dimensions. Data within each gel were normalized (expressed as a percentage of total spot abundance) and routine statistical analyses available within the software package were used to identify unique spots, absent spots, or spots up or down regulated under specified conditions.

### Trypsin Digestion

Following electrophoresis, staining, scanning, spot detection, and match set preparation, proteins of interest were selected and their standard spot numbers entered into a “Cut List.” This “Cut List” was used by the automated spot cutter to select and excise the protein features in order of least to most abundant from one or more gels. Excised gel plugs were deposited into a 96 well plate and transferred to the MassPrep (Waters/Micromass) station. Proteins within the gel plugs were automatically destained, reduced, alkylated, dehydrated, rehydrated and digested with trypsin. The resulting peptides were extracted, cleaned-up, and then deposited into 96 well plates for analysis.

### Q-TOF Analysis

The peptides from each digested spot were separated by capillary liquid chromatography interfaced to an ESI-MS/MS MicroMass Q-TOF micro mass spectrometer. MassLynx 4.0 software package (Waters) was used to identify individual mass spectrograms. Parameters included calculation of charge states and peaks were de-isotoped. The ProteinLynxGlobalServer 1.1 software was used to search Release 43.0 of Swiss-Prot containing 146,720 sequence entries for protein identification using 100 ppm precursor-ion and fragment-ion mass accuracy, modifications included phosphorylation, oxidation of methionine, and cysteines modified with iodoacetamide, 1 missed cleavage and using trypsin. Scores above 100 were generally considered valid identifications, although any identification with a score below 200 was examined carefully, to verify that the spectra included a good number of consecutive “y” ions with high mass accuracy. The number of peptides analyzed and the percentage coverage of the total amino acid sequence was determined for each protein identified. The database was checked for redundancy and inspected for single proteins listed under multiple names. The molecular weight and pI of identified proteins were evaluated and verified relative to the electrophoretic mobility of the protein feature on the 2-dimensional gel.

### Criteria Used for Analysis

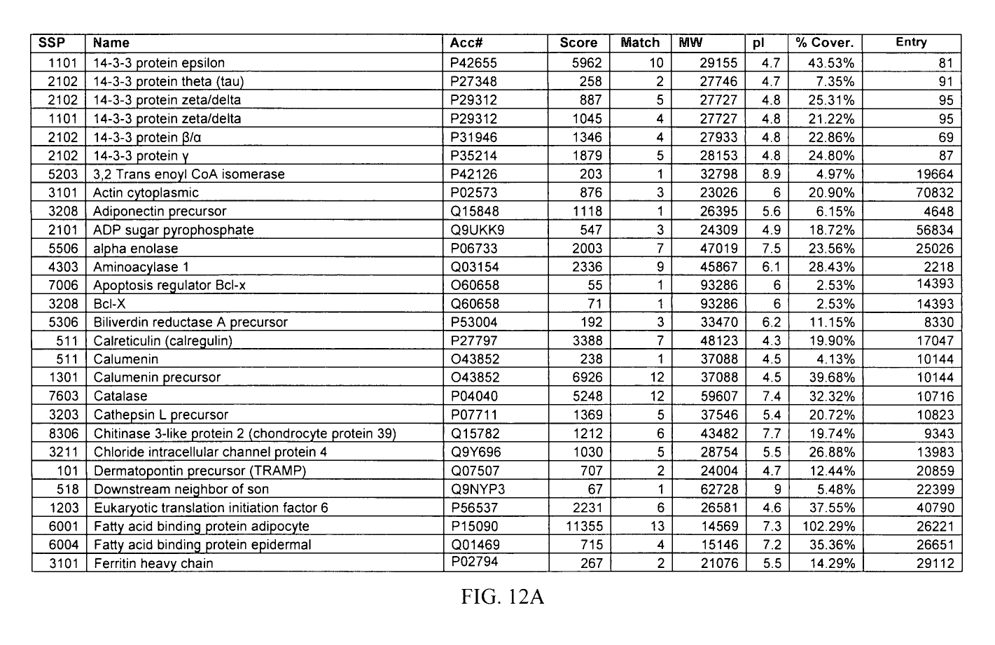

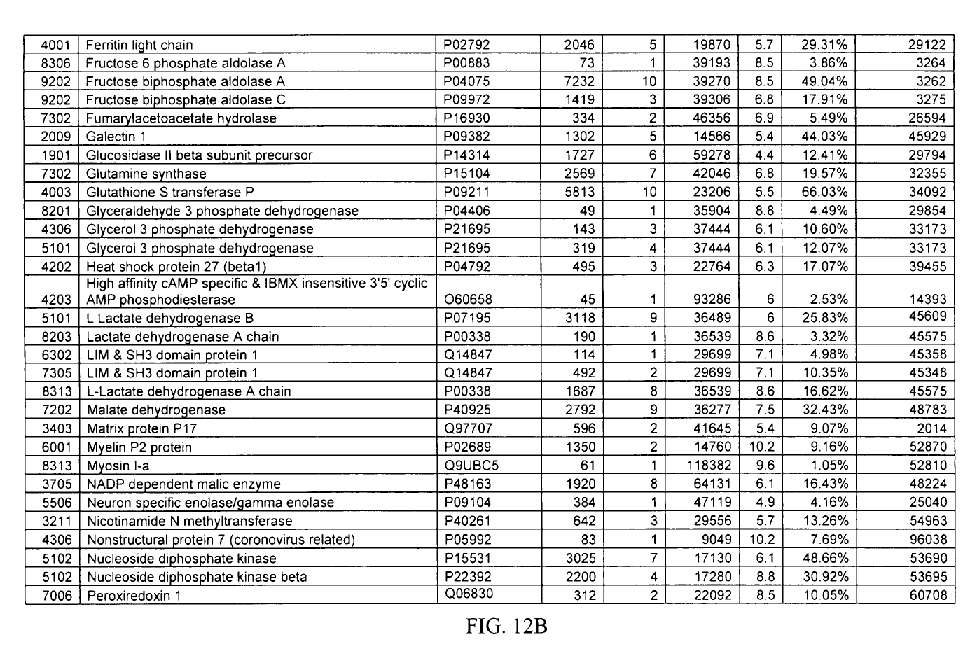

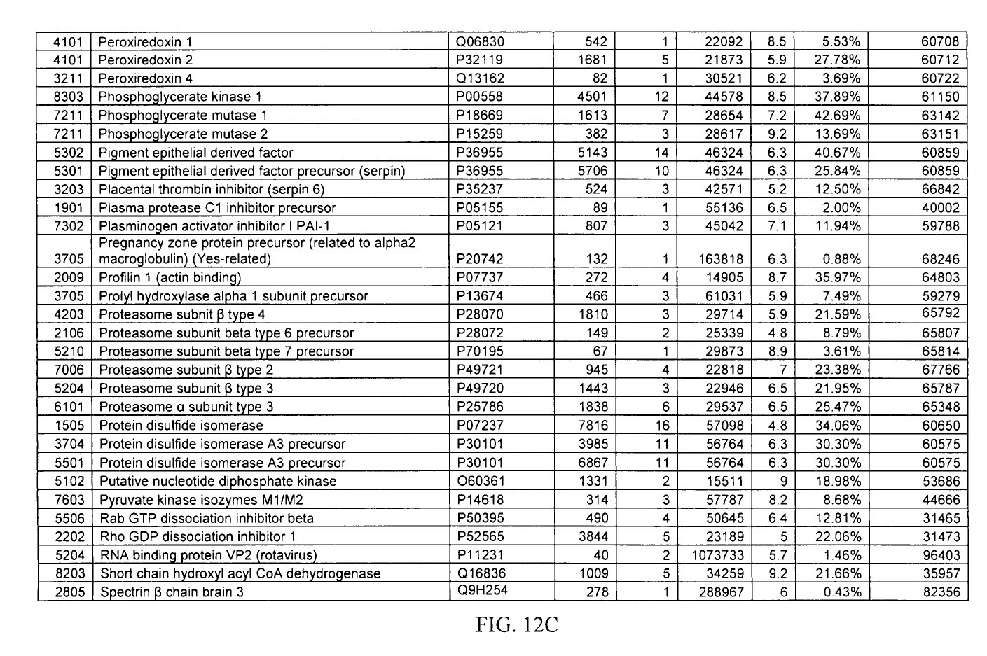

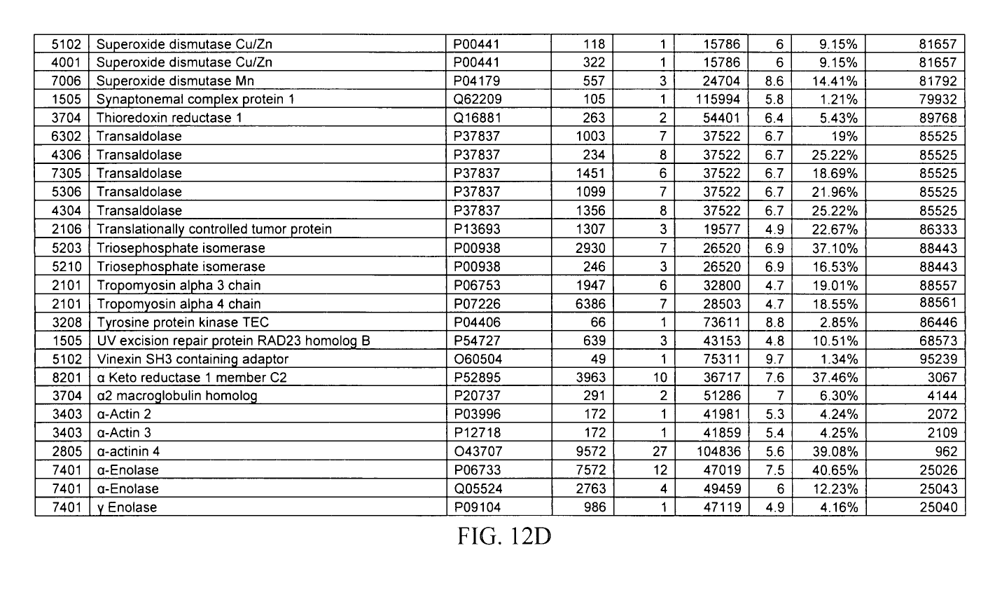

The proteome of the undifferentiated and differentiated human adipose-derived adult stem cells was defined based on the following guidelines: (1) Proteins “induced” or “reduced” during adipogenesis displayed both a 95% significance in comparisons between replicate groups and >2-fold induction or >2-fold reduction with Adipocyte Differentiation (total of 81 features; FIG. 12).

### Affymetrix Oligonucleotide Microarray Gene Expression Analysis

The integrity of total RNA isolated from the uninduced and induced cells was assessed by electrophoresis on the Agilent 2100 Bioanalyzer (Agilent Technologies, Palo Alto, Calif.). Double-stranded cDNA was synthesized from a pool of total RNA with equal aliquots from all four donors under either uninduced or induced culture conditions using a Superscript cDNA Synthesis Kit (Invitrogen, Carlsbad, Calif.) in combination with a T7-(dT)24 primer. Biotinylated cRNA was transcribed in vitro using the GeneChip IVT Labeling Kit (Affymetrix, Santa Clara, Calif.) and purified using the GeneChip Sample Cleanup Module. Ten micrograms of purified cRNA was fragmented by incubation in fragmentation buffer (200 mM Tris-acetate, pH 8.1, 500 mM potassium acetate, 150 mM magnesium acetate) at 94° C. for 35 minutes and chilled on ice. Fragmented biotin-labeled cRNA (6.5 μg) was hybridized to the Human Genome Array (Affymetrix). Arrays were incubated for 16 hours at 45° C. with constant rotation (60 rpm), washed, and stained for 10 minutes at 25° C. with 10 μg/mL streptavidin-R phycoerythrin (Vector Laboratories, Burlingame, Calif.) followed by 3 μg/mL biotinylated goat anti-streptavidin antibody (Vector Laboratories) for 10 minutes at 25° C. Arrays were stained once again with streptavidin-R phycoerythrin for 10 minutes at 25° C., washed, and scanned using a GeneChip Scanner 3000. Pixel intensities were measured, expression signals were analyzed and features extracted using the commercial software package GeneChip Operating Software v.1.2 (Affymetrix). Data mining and statistical analyses were performed with Data Mining Tool v.3.0 (Affymetrix) algorithms. Arrays were globally scaled to a target intensity value of 2500 in order to compare individual experiments. The absolute call (present, marginal, absent) of each gene expression in each sample, as well as the direction of change, and fold change of gene expressions between samples were identified using the above-mentioned software.

### Quantitative Real-Time RT-PCR (qRT-PCR)

Total RNA was purified from tissues using TRIREAGENT® (Molecular Research Center) according to the manufacturer's specifications. Approximately 2 μg of total RNA was reverse transcribed using Moloney Murine Leukemia Virus Reverse Transcriptase (MMLV-RT; Promega, Madison, Wis.), with Oligo dT at 42° C. for 1 hour in a 20 μL reaction. Primers for genes of interest (Table 5) were identified using Primer Express software (Applied Biosystems) and were designed to cross at least one exon/intron boundary. qRT-PCR was performed on diluted cDNA samples with SYBR® Green PCR Master Mix (Applied Biosystems) using the 7900 Real Time PCR system (Applied Biosystems) under universal cycling conditions (95° C. for 10 minutes; 40 cycles of 95° C. for 15 seconds; then 60° C. for 1 minute). All results were normalized relative to a Cyclophilin B expression control. The following sets of primers were used:

TABLE 4

| Gene/ | | | |
|---|---|---|---|
| Pro- | Nucleo- | | |
| tein | tide | Primer | |
| Name | Sequence | Site | Primer Sequence |
| Pro- | NM_000062 | Forward | CAGCTCTCCCACAATCTGAGTTT |
| tease | | bp | (SEQ ID NO: 31) |
| C1 in- | | 1233–1255 | |
| hibi- | | | |
| tor | | | |
| Pro- | NM_000062 | Reverse | CAGCTCTCCCACAATCTGAGTTT |
| tease | | bp | (SEQ ID NO: 32) |
| C1 in- | | 1312–1290 | |
| hibi- | | | |
| tor | | | |
| PAI-1 | NM_006216 | Forward | GGTCCGGAATGTGAACTTTGAG |
| | | bp | (SEQ ID NO: 33) |
| | | 611–632 | |
| PAI-1 | NM_006216 | Reverse | TGTCAATCATATCCCTGGTTTCAT |
| | | bp | (SEQ ID NO: 34) |
| | | 699–676 | |
| PEDF | NM_002615 | Forward | GCAGGCGGTCCTCACTGT |
| | | bp | (SEQ ID NO: 35) |
| | | 1081–1098 | |
| PEDF | NM_002615 | Reverse | AACAAGGATTGCAGCTTCATCTC |
| | | bp | (SEQ ID NO: 36) |
| | | 1170–1148 | |
| Vaspin | NM_173850 | Forward | TCATCGGCCCTACAGAGAAGA |
| | | bp | (SEQ ID NO: 37) |
| | | 373–393 | |
| Vaspin | NM_173850 | Reverse | CACCAGGGCAGCAACACTTA |
| | | bp | (SEQ ID NO: 38) |
| | | 460–441 | |

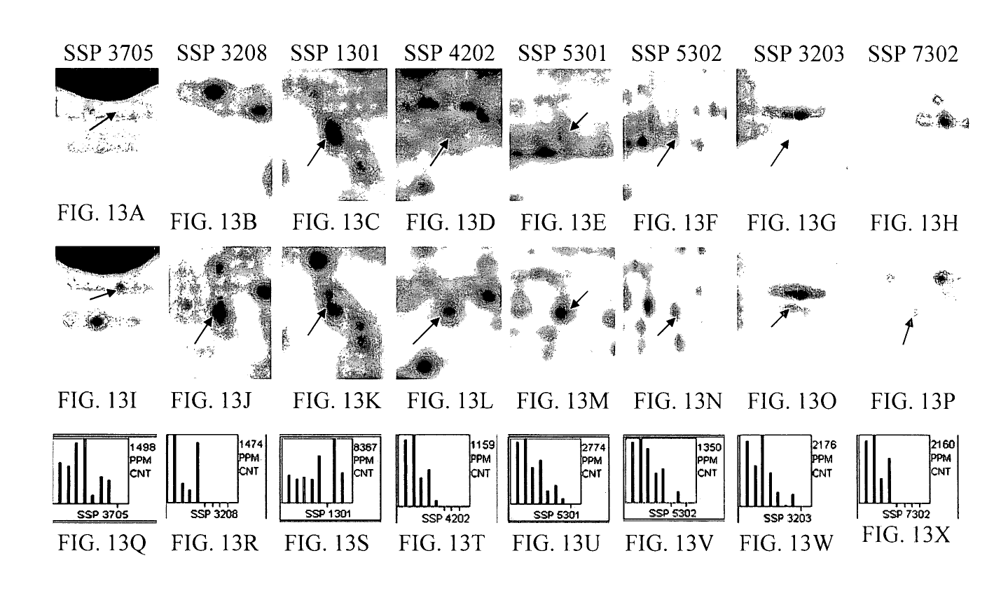

As demonstrated by the data disclosed herein, as depicted in the two-dimensional gels prepared with protein lysates depicted in FIG. 13, there are numerous differing protein features between undifferentiated and differentiated human adipocyte-derived adult stem cells. Changes for representative protein features were evident between two-dimensional gels prepared with protein lysates from undifferentiated and differentiated adipose-derived adult stem cells. Specific differences noted in FIG. 13 include pregnancy zone protein precursor (SSP 3705), adiponectin precursor (SSP 3208), calumenin precursor (1301), heat shock protein 27 (beta 1) (SSP 4202), pigment epithelial derived factor precursor (serpin) (SSP 5301), pigment epithelial derived factor (5302), placental thrombin inhibitor (serpin 6) (SSP 3203), and plasminogen activator inhibitor I PAI-1 (SSP 7302). The arrows in FIG. 13 indicate the location of the protein features. The bar graph indicates relative abundance of the spot on the undifferentiated gels versus the differentiated cells.

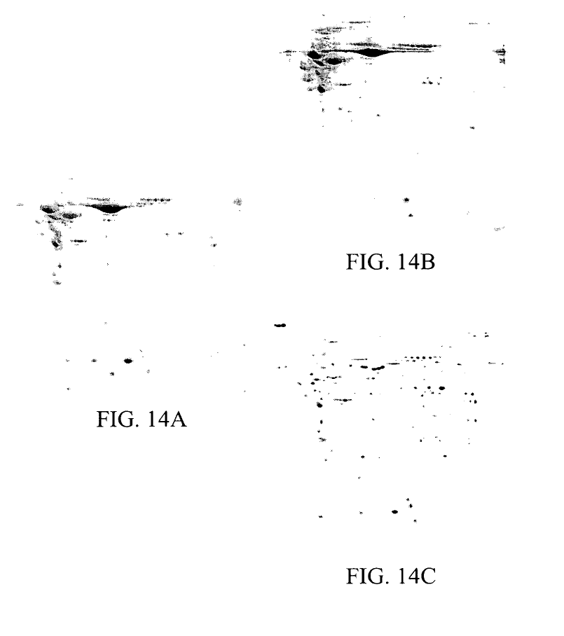

The different in secreted protein expression in undifferentiated versus differentiated adipose-derived adult stem cells is demonstrated in FIG. 14. Two-dimensional PAGE was performed with protein lysates prepared from human adipose-derived adult stem cells in the undifferentiated and differentiated condition nine days following induction. The gels were stained with Sypro Ruby. FIG. 14 depicts representative gels from each condition as well as the master composite prepared based on features conserved on replicate gels prepared from protein extracts obtained from the four individual donors.

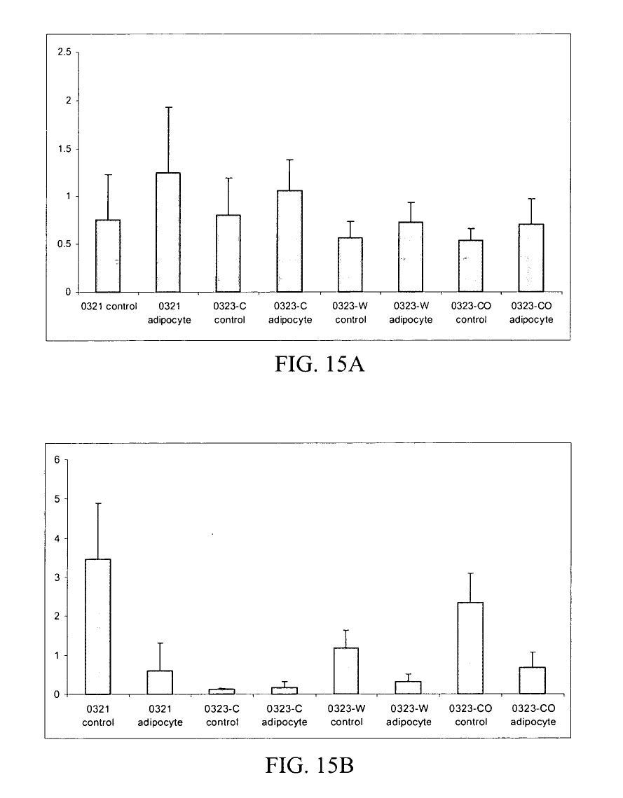

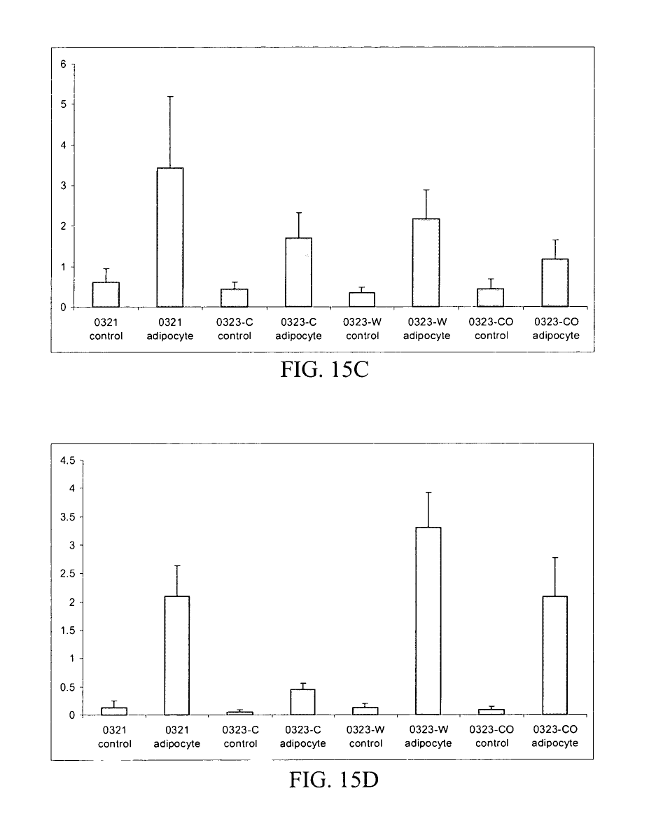

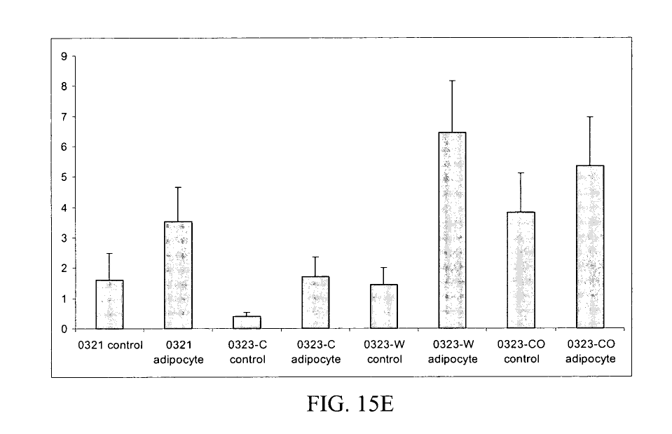

The correlation between protein expression and genomic expression were assessed by quantitative real time PCR. FIG. 15A depicts quantitative real time PCR results of protease C1 inhibitor normalized to cyclophilin B for control (undifferentiated) and differentiated human adipose-derived adult stem cells from four individual donors. Values are the mean±S.D. for triplicate determinations for each donor sample. FIG. 15B depicts quantitative real time PCR results of plasminogen activator inhibitor-1 (PAI-1) normalized to cyclophilin B for control (undifferentiated) and differentiated human adipose-derived adult stem cells from four individual donors. Values are the mean±S.D. for triplicate determinations for each donor sample. FIG. 15C depicts quantitative real time PCR results of pigmented epithelial derived factor (PEDF) normalized to cyclophilin B for control (undifferentiated) and differentiated human adipose-derived adult stem cells from four individual donors. Values are the mean±S.D. for triplicate determinations for each donor sample. FIG. 15D depicts quantitative real time PCR results of crystallin αB normalized to cyclophilin B for control (undifferentiated) and differentiated human adipose-derived adult stem cells from four individual donors. Values are the mean±S.D. for triplicate determinations for each donor sample. FIG. 15E depicts quantitative real time PCR results of heat shock protein 27 normalized to cyclophilin B for control (undifferentiated) and differentiated human adipose-derived adult stem cells from four individual donors. Values are the mean±S.D. for triplicate determinations for each donor sample.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of identifying a differentiated adipose-derived adult stem cell, said method comprising comparing a proteomic profile of a first adipose-derived adult stem cell to a proteomic profile of a second adipose-derived adult stem cell, wherein said proteomic profile of said first adipose-derived adult stem cell comprises a protein that is specific for said first adipose-derived adult stem cell and is not upregulated in the proteomic profile of said second adipose-derived adult stem cell, thereby identifying a differentiated adipose-derived adult stem cell.

2. The method of claim 1, wherein said adipose-derived adult stem cell is a human adipose-derived adult stem cell.

3. The method of claim 1, wherein said protein is selected from the group consisting of a metabolism-related protein, a heat shock protein, a redox protein, a cytoskeletal protein, a serine protease inhibitor protein, and a protein degradation-related protein.

4. The method of claim 1 wherein said proteomic profile comprises at least two proteins specific for said differentiated adipose-derived adult stem cell.

5. The method of claim 1, wherein said protein specific for said differentiated adipose-derived adult stem cell is upregulated about 2-fold compared to said second adipose-derived adult stem cell.

6. The method of claim 5, wherein said protein is selected from the group consisting of fatty acid binding protein-adipocyte, heat shock protein 20-like protein, heat shock protein β, heat shock protein 20, heat shock protein 27, heat shock protein 60, plasminogen activator inhibitor-1, pigmented epidermal derived factor, placental thrombin inhibitor, pregnancy zone protein, and protease C1 inhibitor.

7. A method of identifying a differentiated adipose-derived adult stem cell, said method comprising comparing a proteomic profile of a first adipose-derived adult stem cell to a proteomic profile of a second adipose-derived adult stem cell, wherein said proteomic profile of said first adipose-derived adult stem cell comprises a protein that is specific for said first adipose-derived adult stem cell and is not downregulated in the proteomic profile of said second adipose-derived adult stem cell, thereby identifying a differentiated adipose-derived adult stem cell.

8. The method of claim 7, wherein said adipose-derived adult stem cell is a human adipose-derived adult stem cell.

9. The method of claim 7, wherein said protein is selected from the group consisting of a metabolism-related protein, a heat shock protein, a redox protein, a cytoskeletal protein, a serine protease inhibitor protein, and a protein degradation-related protein.

10. The method of claim 7 wherein said proteomic profile comprises at least two proteins specific for said differentiated adipose-derived adult stem cell.

11. The method of claim 7, wherein said protein is selected from the group consisting of stathmin, elfin, LIM, and SH3 domain protein 1.

12. A method of distinguishing an undifferentiated adipose-derived adult stem cell from an differentiated adipose-derived adult stem cell, said method comprising comparing a proteomic profile of said undifferentiated adipose-derived adult stem cell to a proteomic profile of a differentiated adipose-derived adult stem cell, wherein said proteomic profile of said differentiated adipose-derived adult stem cell comprises a protein that is specific for said differentiated adipose-derived adult stem cell, further wherein said undifferentiated adipose-derived adult stem cell does not detectably express said protein that is specific for said differentiated adipose-derived adult stem, thereby distinguishing an undifferentiated adipose-derived adult stem cell from a differentiated adipose-derived adult stem cell.

13. The method of claim 12, wherein said adipose-derived adult stem cell is a human adipose-derived adult stem cell.

14. The method of claim 12, wherein said protein is selected from the group consisting of a metabolism-related protein, a heat shock protein, a redox protein, a cytoskeletal protein, a serine protease inhibitor protein, and a protein degradation-related protein.

15. The method of claim 12, wherein said proteomic profile comprises at least two proteins specific for said differentiated adipose-derived adult stem cell.

16. The method of claim 12, wherein said protein specific for said differentiated adipose-derived adult stem cell is upregulated about 2-fold compared to said undifferentiated adipose-derived adult stem cell.

17. The method of claim 12, wherein said protein is selected from the group consisting of fatty acid binding protein-adipocyte, heat shock protein 20-like protein, heat shock protein β, heat shock protein 20, heat shock protein 27, heat shock protein 60, plasminogen activator inhibitor-1, pigmented epidermal derived factor, placental thrombin inhibitor, pregnancy zone protein, and protease C1 inhibitor.

18. A method of selecting an adipose-derived adult stem cell from a population of adipose-derived adult stem cells, said method comprising comparing a proteomic profile of said adipose-derived adult stem cell to a proteomic profile of said population of adipose-derived adult stem cells, wherein said proteomic profile of said adipose-derived adult stem cell comprises a protein that is specific for said adipose-derived adult stem cell and is not upregulated in said proteomic profile of said population of adipose-derived adult stem cells, thereby selecting an adipose-derived adult stem cell from a population of adipose-derived adult stem cells.

19. The method of claim 18, wherein said adipose-derived adult stem cell is a human adipose-derived adult stem cell.

20. The method of claim 18, wherein said protein is selected from the group consisting of a metabolism-related protein, a heat shock protein, a redox protein, a cytoskeletal protein, a serine protease inhibitor protein, and a protein degradation-related protein.

21. The method of claim 18, wherein said proteomic profile comprises at least two proteins specific for said adipose-derived adult stem cell.

22. The method of claim 18, wherein said protein specific for said adipose-derived adult stem cell is upregulated about 2-fold compared to said population of adipose-derived adult stem cells.

23. The method of claim 22, wherein said protein is selected from the group consisting of fatty acid binding protein-adipocyte, heat shock protein 20-like protein, heat shock protein β, heat shock protein 20, heat shock protein 27, heat shock protein 60, plasminogen activator inhibitor-1, pigmented epidermal derived factor, placental thrombin inhibitor, pregnancy zone protein, and protease C1 inhibitor.

24. A method of identifying a compound that differentiates an adipose-derived adult stem cell, said method comprising contacting said adipose-derived adult stem cell with said compound, comparing a proteomic profile of said adipose-derived adult stem cell so contacted to a proteomic profile of an adipose-derived adult stem cell not contacted with said compound, wherein said proteomic profile of said adipose-derived adult stem cell so contacted comprises a protein that is specific for a differentiated adipose-derived adult stem cell and is not upregulated in said adipose-derived adult stem cell not contacted with said compound, thereby identifying a compound that differentiates an adipose-derived adult stem cell.

25. The method of claim 24, wherein said adipose-derived adult stem cell is a human adipose-derived adult stem cell.

26. The method of claim 24, wherein said protein is selected from the group consisting of a metabolism-related protein, a heat shock protein, a redox protein, a cytoskeletal protein, a serine protease inhibitor protein, and a protein degradation-related protein.

27. The method of claim 24, wherein said proteomic profile comprises at least two proteins specific for said differentiated adipose-derived adult stem cell.

28. The method of claim 24, wherein said protein specific for said differentiated adipose-derived adult stem cell is upregulated.

29. The method of claim 28, wherein said protein is selected from the group consisting of fatty acid binding protein-adipocyte, heat shock protein 20-like protein, heat shock protein β, heat shock protein 20, heat shock protein 27, heat shock protein 60, plasminogen activator inhibitor-1, pigmented epidermal derived factor, placental thrombin inhibitor, pregnancy zone protein, and protease C1 inhibitor.

30. A compound identified by the method of claim 24.


SEQUENCE LISTING (69 sequences)

SEQ ID NO: 1 - protein, 13 aa, Homo sapiens
KEAAENSLVAYKA

SEQ ID NO: 2 - protein, 9 aa, Homo sapiens
KDVIEEYFC

SEQ ID NO: 3 - protein, 14 aa, Homo sapiens
KNIEDVIAQGIGKL

SEQ ID NO: 4 - protein, 13 aa, Homo sapiens
REFSGYVESGLKT

SEQ ID NO: 5 - protein, 16 aa, Homo sapiens
KAQAELVGTADEATRA

SEQ ID NO: 6 - protein, 11 aa, Homo sapiens
KDGYNYTLSKT

SEQ ID NO: 7 - protein, 14 aa, Homo sapiens
RYISPDQLADLYKS

SEQ ID NO: 8 - protein, 13 aa, Homo sapiens
RYALYDATYETKE

SEQ ID NO: 9 - protein, 13 aa, Homo sapiens
KQYDTYGEEGLKD

SEQ ID NO: 10 - protein, 14 aa, Homo sapiens
RIASLEVENQSLRG

SEQ ID NO: 11 - protein, 20 aa, Homo sapiens
RTIGTGLVTNTLAMTEEEKN

SEQ ID NO: 12 - protein, 10 aa, Homo sapiens
KIFEYETQRR

SEQ ID NO: 13 - protein, 14 aa, Homo sapiens
GVQVETISPGDGRT

SEQ ID NO: 14 - protein, 16 aa, Homo sapiens
RNDLSPTTVMSEGARN

SEQ ID NO: 15 - protein, 12 aa, Homo sapiens
PPYTVVYFPVRG

SEQ ID NO: 16 - protein, 14 aa, Homo sapiens
RLFVSGACDASAKL

SEQ ID NO: 17 - protein, 11 aa, Homo sapiens
RMFLSFPTTKT

SEQ ID NO: 18 - protein, 17 aa, Homo sapiens
KAMGIMNSFVNDIFERI

SEQ ID NO: 19 - protein, 11 aa, Homo sapiens
KFFDANYDGKD

SEQ ID NO: 20 - protein, 15 aa, Homo sapiens
KLNQPPEDGISSVKF

SEQ ID NO: 21 - protein, 15 aa, Homo sapiens
RALGQNPTNAEVLKV

SEQ ID NO: 22 - protein, 18 aa, Homo sapiens
REMQDVDLAEVKPLVEKG

SEQ ID NO: 23 - protein, 11 aa, Homo sapiens
KEGEFVAQFKF

SEQ ID NO: 24 - protein, 13 aa, Homo sapiens
RLAAIAESGVERQ

SEQ ID NO: 25 - protein, 12 aa, Homo sapiens
KVIMDYESLEKA

SEQ ID NO: 26 - protein, 11 aa, Homo sapiens
KLNLDSIIGRL

SEQ ID NO: 27 - protein, 11 aa, Homo sapiens
RFTEYETQVKV

SEQ ID NO: 28 - protein, 14 aa, Homo sapiens
RDMNQVLDAYENKK

SEQ ID NO: 29 - protein, 15 aa, Homo sapiens
KTAFQEALDAAGDKL

SEQ ID NO: 30 - protein, 12 aa, Homo sapiens
KLGFEDGSVLKQ

SEQ ID NO: 31 - DNA, 23 bp, Artificial Sequence
  Chemically Synthesized PCR Primer
cagctctcccacaatctgagttt

SEQ ID NO: 32 - DNA, 23 bp, Artificial Sequence
  Chemically Synthesized PCR Primer
cagctctcccacaatctgagttt

SEQ ID NO: 33 - DNA, 22 bp, Artificial Sequence
  Chemically Synthesized PCR Primer
ggtccggaatgtgaactttgag

SEQ ID NO: 34 - DNA, 24 bp, Artificial Sequence
  Chemically Synthesized PCR Primer
tgtcaatcatatccctggtttcat

SEQ ID NO: 35 - DNA, 18 bp, Artificial Sequence
  Chemically Synthesized PCR Primer
gcaggcggtcctcactgt

SEQ ID NO: 36 - DNA, 23 bp, Artificial Sequence
  Chemically Synthesized PCR Primer
aacaaggattgcagcttcatctc

SEQ ID NO: 37 - DNA, 21 bp, Artificial Sequence
  Chemically Synthesized PCR Primer
tcatcggccctacagagaaga

SEQ ID NO: 38 - DNA, 20 bp, Artificial Sequence
  Chemically Synthesized PCR Primer
caccagggcagcaacactta

SEQ ID NO: 39 - protein, 10 aa, Homo sapiens
KDIMAEIYKN

SEQ ID NO: 40 - protein, 13 aa, Homo sapiens
RYALYDASFETKE

SEQ ID NO: 41 - protein, 12 aa, Homo sapiens
RMSVLPTPASRR

SEQ ID NO: 42 - protein, 12 aa, Homo sapiens
RQLSSGVSEIRH

SEQ ID NO: 43 - protein, 12 aa, Homo sapiens
RQLSSGVSEIRH

SEQ ID NO: 44 - protein, 12 aa, Homo sapiens
RLFDQAFGLPRL

SEQ ID NO: 45 - protein, 12 aa, Homo sapiens
RQLSSGVSEIRH

SEQ ID NO: 46 - protein, 16 aa, Homo sapiens
RLEGAEINKSLLALKE

SEQ ID NO: 47 - protein, 11 aa, Homo sapiens
RNEASDLLERL

SEQ ID NO: 48 - protein, 12 aa, Homo sapiens
RTIAQDYGVLKA

SEQ ID NO: 49 - protein, 12 aa, Homo sapiens
RQLSSGVSEIRH

SEQ ID NO: 50 - protein, 12 aa, Homo sapiens
RLFDQAFGLPRL

SEQ ID NO: 51 - protein, 12 aa, Homo sapiens
RSMSLNIGGAKG

SEQ ID NO: 52 - protein, 12 aa, Homo sapiens
RFSGTWYAMAKK

SEQ ID NO: 53 - protein, 16 aa, Homo sapiens
KGDGPVQGIINFEQKE

SEQ ID NO: 54 - protein, 17 aa, Homo sapiens
RTLNQLGTPQDSPELRQ

SEQ ID NO: 55 - protein, 10 aa, Homo sapiens
KFLIDGFPRN

SEQ ID NO: 56 - protein, 16 aa, Homo sapiens
KGVDEVTIVNILTNRS

SEQ ID NO: 57 - protein, 14 aa, Homo sapiens
KLVIPSELGYGERG

SEQ ID NO: 58 - protein, 12 aa, Homo sapiens
RQLSSGVSEIRH

SEQ ID NO: 59 - protein, 16 aa, Homo sapiens
KQMEQISQFLQAAERY

SEQ ID NO: 60 - protein, 15 aa, Homo sapiens
RQGNMTAALQAALKN

SEQ ID NO: 61 - protein, 16 aa, Homo sapiens
KAQAELVGTADEATRA

SEQ ID NO: 62 - protein, 14 aa, Homo sapiens
KSQVVAGTNYFIKV

SEQ ID NO: 63 - protein, 12 aa, Homo sapiens
KYVECSALTQKG

SEQ ID NO: 64 - protein, 13 aa, Homo sapiens
RLLVPYLMEAIRL

SEQ ID NO: 65 - protein, 12 aa, Homo sapiens
RDIDEVSSLLRT

SEQ ID NO: 66 - protein, 14 aa, Homo sapiens
KVAGQDGSVVQFKI

SEQ ID NO: 67 - protein, 16 aa, Homo sapiens
KLYTLVLTDPDAPSRK

SEQ ID NO: 68 - protein, 12 aa, Homo sapiens
RMSVLPTPASRR

SEQ ID NO: 69 - protein, 11 aa, Homo sapiens
RNEASDLLERL